(19) United States
(12) Patent Application Publication
(10) Pub. No.: US 2017/0079519 A1
(43) Pub. Date: Mar. 23, 2017

(54) BIOPSY DEVICES, SYSTEMS, AND METHODS FOR USE

(71) Applicant: The Board of Trustees of the Leland Stanford Junior University, Palo Alto, CA (US)

(72) Inventors: Arthur Wai Sung, Atherton, CA (US); Ryan J.F. Van Wert, San Francisco, CA (US); Benjamin Goggio Cohn, Oakhurst, CA (US); Vidya Bhat, Palo Alto, CA (US); Jennifer Fasman, Redwood City, CA (US); Neeraj Kumar, Noida (IN); Hong Vo, San Francisco, CA (US)

(21) Appl. No.: 15/283,066

(22) Filed: Sep. 30, 2016

Related U.S. Application Data
(63) Continuation of application No. PCT/US15/24184, filed on Apr. 2, 2015.
(60) Provisional application No. 61/974,003, filed on Apr. 2, 2014.
(60) Provisional application No. 62/004,228, filed on May 29, 2014.
(60) Provisional application No. 62/058,102, filed on Oct. 1, 2014.

(51) Int. Cl. A61B 1/267; A61B 1/012; A61B 8/00; A61B 8/12; A61B 10/02

(52) U.S. Cl. CPC A61B 1/2676; A61B 8/12; A61B 10/0283; A61B 8/4281; A61B 1/012

(57) ABSTRACT

Apparatus, systems, and methods are provided for performing a biopsy within a patient's lung using an access sheath or catheter including a distal portion sized for introduction into an airway of a lung. An ultrasound imaging device is deployable from the distal portion for imaging tissue adjacent the body lumen, and a needle or other biopsy may be advanced from the distal portion into surrounding tissue, e.g., to obtain a tissue sample.

### RELATED APPLICATION DATA

The present application is a continuation of co-pending International Application PCT/US2015/024184, filed Apr. 2, 2015, which claims benefit of provisional application Ser. No. 61/974,003, filed Apr. 2, 2014, 62/004,228, filed May 29, 2014, and 62/058,102, filed Oct. 1, 2014, the entire disclosures of which are expressly incorporated by reference herein.

### FIELD OF THE INVENTION

The present invention relates to apparatus, systems, and methods for accessing body lumens within a patient's body, e.g., to perform a biopsy, and more particularly to biopsy devices and systems and methods for using such devices to access a patient's lung and/or to perform a biopsy within a patient's lung.

### BACKGROUND

Lung cancer is the leading cause of cancer mortality in the United States. Early diagnosis is key: survival rates rise from 17% to 52% if the cancer is detected at an early stage. Until recently, no effective framework for lung cancer screening was in place. However, a recent landmark study showed a dramatic reduction in lung cancer mortality by screening at-risk individuals using low-dose CT scans. These findings prompted the United States Preventative Services Task Force (USPSTF) to recommend annual screening for high-risk individuals.

Lung biopsy is a medical procedure used to obtain a sample of tissue in order to diagnose various diseases. Current methods for obtaining a lung biopsy include surgical procedures, image-guided biopsy or aspiration, or biopsy via a bronchoscope. In many instances, there is a desire to obtain tissue from a specific area of interest in the lungs, for example, within a lung nodule. Current technologies facilitate navigation of a bronchoscope to a region of interest with various degrees of accuracy, but there remain limitations to such procedures. Many limitations are due to the fact that navigation methods utilize fixed computed tomography images, but do not account for real-time variations in functional lung anatomy including normal respiration patterns. In addition, bronchoscopes may have design limitations that limit the size of biopsy and/or imaging instruments that may be introduced because of the relatively small working channel available to introduce instruments through the bronchoscope to an area of interest.

Accordingly, apparatus, systems, and methods that facilitate accessing a patient's lung and/or facilitate performing a biopsy within a lung would be useful.

### SUMMARY

The present invention is directed to apparatus, systems, and methods for accessing body lumens within a patient's body, e.g., to perform a biopsy, and more particularly to biopsy devices and systems and methods for using such devices to access a patient's lung and/or to perform a biopsy within a patient's lung. For example, the devices and methods herein may facilitate directing biopsy tools in real-time to an area of interest, e.g., using imaging tools and/or a working channel that allows relatively larger instruments to be introduced into the area of interest.

In an exemplary embodiment, an access sheath or catheter is provided that is configured to fit over at least part of a bronchoscope and/or other guide instrument and that may be introduced through one or more airways within a patient's lung to reach a target location, e.g., within the bronchial tree close to a region of interest, e.g., a target biopsy site. Generally, the sheath includes an elongate tubular member including a proximal end, a distal end sized for introduction into a patient's body, and one or more lumens extending therebetween. In exemplary embodiments, the guide instrument may include one or more locatable guides, e.g., components of electromagnetic navigation systems, radio-opaque wires advanced using fluoroscopic guidance, and/or other navigation systems that may be used to guide the guide instrument to a desired location in the airway.

During use, with the sheath placed over a bronchoscope and/or other guide instrument, the bronchoscope/guide instrument and sheath may be introduced into a patient's lung until a distal portion thereof has reached a desired location in an airway. The bronchoscope and/or guide instrument may then be removed, leaving the sheath in place. In this way, the sheath may serve as a conduit through which other devices or objects of interest may be introduced into the region of interest. The sheath may be substantially rigid, semi-rigid, or flexible along its length, and optionally, may have a variable diameter, e.g., may be folded on itself at one or more regions to accommodate various diameters of the instrument(s) it surrounds.

In another exemplary embodiment, an expandable access sheath is provided that is configured to be introduced through a working channel of a bronchoscope, e.g., in a contracted or collapsed configuration, and deployable within an airway, e.g., in an expanded configuration. This may be achieved in various ways. For example, the sheath may be resiliently biased to a generally cylindrical or other expanded configuration, and may be folded on itself, rolled, and the like to adopt the contracted configuration, e.g., using one or more creases, pleats, folds, weakened regions, and the like to facilitate compressing the sheath. In addition or alternatively, the material of the sheath may be pre-stressed or may include spring or shape-memory materials that expand upon deployment towards the expanded configuration but may be resiliently compressed to the contracted configuration for delivery. In another embodiment, the sheath may be formed from flexible material that may expand in the cross-sectional dimension when an internal portion of the sheath, e.g., an annular wall or lumen, is subjected to increased pressure from a gas or liquid. In still another embodiment, the sheath may include a plurality of support struts that may reorient themselves between the contracted and expanded configurations. For example, the struts may initially be oriented substantially parallel to a longitudinal axis of the sheath to adopt the contracted configuration and, when deployed, transition to be substantially perpendicular to the longitudinal axis and/or peripherally to expand the wall of the sheath towards the expanded configuration.

During use, the sheath may be inserted into the bronchoscope in the contracted configuration before introduction, and the sheath and bronchoscope may be introduced into the patient's body substantially simultaneously. Alternatively, a distal end of the bronchoscope may be introduced first and then a distal portion of the sheath may be advanced through the bronchoscope until disposed adjacent the distal end. The distal portion of the sheath may remain adjacent the distal end of the bronchoscope or it may be advanced beyond the distal end, e.g., into more distal airways within the lung. Such advancement may be guided by any of the navigation methods described herein, if desired, with or without an associated guide instrument. Once the desired position has been reached, the bronchoscope and any associated guide instruments may be withdrawn, leaving the sheath in place. The sheath may automatically expand upon withdrawal of the bronchoscope or may be selectively expanded by the user.

Optionally, the distal portion of the sheath may include one or more anchoring elements to secure the distal portion relative to a desired location in the airway. In an exemplary embodiment, the anchoring element may include a balloon or other expandable member, which may be inflated or otherwise expanded to be in apposition with the airway walls. Optionally, the balloon surface may be smooth or contain various surface variations configured to increase the purchase of the balloon to the airway wall. Such surface variations may include one or more ridges, bumps, or other macro or microscopic patterns. In addition or alternatively, the balloon surface may be coated with an adhesive substance and/or with a viscous or “sticky” liquid. In an alternative embodiment, the anchoring element may include one or more hooks and/or needles, which may or may not be barbed. Such hooks and/or needles may be deployed by slightly retracting the sheath such that the anchoring elements contact the airway wall, or by other mechanical actuators, such as a pull wire, pull string, spring mechanism, and the like. Removal may be achieved through similar mechanisms.

Once deployed, any of the sheaths herein may be used to introduce one or more instruments together, sequentially, or in other combinations, e.g., a desired biopsy device, imaging device, and the like to achieve a desired task. Exemplary instruments may include one or more biopsy forceps, biopsy needles, biopsy brushes, fiber optic cameras, infrared cameras, microscopic visualization devices, other tissue sampling devices, ultrasound imaging devices including radial or convex endobronchial ultrasound (“EBUS”) devices, and optical computed tomography imaging devices.

In an exemplary embodiment, a system may be provided that includes an access sheath or delivery catheter in combination with one or both of a real-time imaging device and a directable biopsy device, which may be deployed near a region of interest in the airway. In an exemplary device, the imaging device may be a radial endobronchial ultrasound (EBUS) probe, and the biopsy device may be a biopsy needle. The sheath may include one or more channels or lumens extending between proximal and distal regions of the sheath, and including one or more ports or outlets in the distal portion. For example, the sheath may include an instrument lumen including a ramped surface adjacent an outlet in the distal portion, e.g., to direct a biopsy device or other instrument deployed from the outlet laterally relative to the sheath, e.g., to a desired location in the lung parenchyma.

In an exemplary embodiment, the biopsy needle is a hollow needle attached to or otherwise carried on a hollow conduit or other shaft that travels through the instrument lumen to a proximal portion of the sheath. The distal end of the shaft may then be attached to a needle for purposes of aspiration during a biopsy procedure. During use, the needle and radial EBUS probe may be rotated by the operator to select the region of interest around the airway. The needle may be made of a flexible material, such as Nitinol, silicone, other flexible plastic or pre-stressed material. The radial EBUS probe includes one or more ultrasound transducers, which may or may not be encapsulated with a balloon, which may be filled with an ultrasound-conductive substance, in order to secure the probe against the airway wall and/or enhance acoustically coupling the transducer(s) with adjacent tissue during a biopsy procedure.

In an alternate embodiment, a catheter may be provided that includes a conduit attached to a needle such that the needle exits from a distal portion of the catheter at a desired angle. During use, a physician or other user of the catheter may control the advancement and retraction of the needle and/or the rotational orientation of the needle. The catheter may include one or more clips or other connectors, which may be used to affix the catheter to an imaging device, such as a radial EBUS probe. The connector(s) may permit rotational motion of the catheter relative to the ultrasound probe or may rotationally fix the catheter and probe to one another.

In yet an alternate embodiment, any of the sheaths or catheters may include a balloon or other expandable member on the distal portion, e.g., proximal to the outlet for the biopsy needle. During use, the balloon may be inflated to isolate a region of the airway, e.g., to permit the distal airway to be filled with ultrasound-conducting fluid, such as water or saline solution, via a lumen of the sheath. When the balloon is inflated, the fluid may be substantially isolated from the rest of the airways. The fluid may be delivered through the same lumen used to deliver other instruments or an additional lumen may be provided in the sheath to deliver the fluid and/or to aspirate air or other fluid within the airway.

For example, in yet another exemplary embodiment, the balloon on the distal portion of the sheath may be used to collapse a segment of the lung by first creating a seal within the airway at the position of the balloon and then applying a suction force to remove air from the region beyond the balloon. The suction may or may not be applied using one of the other lumens, e.g., a working or instrument lumen, or an additional aspiration lumen may be provided in the sheath for this purpose. Such collapse may facilitate biopsy, ablation, cryotherapy, and/or other diagnostic or therapeutic procedure in the collapsed segment of the lung.

In yet another embodiment, a biopsy device is provided that includes a hollow flexible or rigid catheter with real-time imaging capability that includes an imaging device, such as an ultrasound probe. In an exemplary embodiment, the catheter may include a lumen extending between proximal and distal ends of the catheter that contain the electrical wiring for operating the ultrasound probe, and a structural component capable of manipulating the position of the ultrasound probe relative to the catheter. In exemplary embodiments, the lumen may have a variety of cross-sectional shapes, e.g., circular, oval, rectangular, concave, and the like, the lattermost may be configured to match the inner or outer contour of the catheter. Once positioned in an airway adjacent to a region of interest to be sampled, the ultrasound probe may be advanced relative to the catheter. In this way, the catheter may be used to deploy a biopsy instrument to the region of interest to obtain a desired sample under real-time visualization with the ultrasound probe.

In accordance with another embodiment, a biopsy device is provided that includes a hollow flexible or rigid catheter with real-time imaging capability and a biopsy device. In an exemplary embodiment, the catheter may include an instrument lumen extending between proximal and distal ends of the catheter for receiving, positioning, deploying, and/or otherwise manipulating the biopsy device. In exemplary embodiments, the instrument lumen may have a variety of cross-sectional shape, e.g., circular, oval, rectangular, concave, and the like, the lattermost to match the inner or outer contour of the catheter. Once positioned in an airway adjacent to a region of interest to be sampled, the biopsy device may be advanced relative to the catheter. In this way, the catheter may be used to deploy an imaging instrument, such as an ultrasound probe to the region of interest to facilitate real-time visualization of the biopsy device during the sampling process. An exemplary biopsy device may be formed from a shape memory material that deploys to a semi-circular shape to advance into the area of interest.

In accordance with yet another embodiment, a biopsy device is provided that includes a plurality of segments formed from material whose rigidity and/or stiffness may be modified when energy or other modifying force is applied. Such materials include piezoelectric materials, heat-sensitive materials, and/or any other material subject to material property change when electromagnetic energy or other force is applied.

In accordance with still another embodiment, a fiducial marker is provided that includes at least two parts. The first part is an object made of a biocompatible material of sufficient size and firmness to be palpated through a desired segment of tissue. The second is a visible light source of sufficient intensity to permeate a desired segment of tissue.

In one embodiment, the fiducial marker may include a biocompatible metal segment and an LED light source. The LED light source may powered by a battery included with the fiducial marker, of from an alternate power source, including an external power source, which may deliver power to the marker via wired or wirelessly transmitted electromagnetic energy. The light source may only become apparent with the application of external electromagnetic energy to the region of interest.

In accordance with another embodiment, an ultrasound probe is provided that includes at least one transducer which may be configured to operate in at least two dimensions or modes in order to obtain a desired image. For example, the transducer may be operated in a first mode where the transducer is rotated about a central axis in order to produce radial ultrasound image slices. In addition or alternatively, the transducer may be operated in a second mode where the transducer is directed in a cyclic manner axially along the central axis, which, when processed by an algorithm known to one skilled in the art, may produce a two dimensional image of the area of interest. Such a linear image may be used for real-time visualization of, for example, a biopsy device or other instrument, which may be advanced in the same plane as the ultrasound transducer.

When utilizing such an ultrasound device capable of sequentially producing both radial and linear images, the user may wish to select an area of interest on the radial images, to be subsequently visualized using the linear mode. In such cases, the user interface and device functionality may permit the user to mark the area of interest on the radial image. In turn, when switched to linear mode, the ultrasound transducer is aligned with that area of interest thus forming a linear image of the area of interest.

In any of the embodiments described herein, there may be digitally produced guide lines or other indicators, e.g., presented on a display to the user after processing and/or analysis by a controller coupled to the transducer, to assist the user in advancing a biopsy instrument under image-guidance. Such guiding indicators may be produced based on actual or calculated trajectories as by the user or through calculations performed by the device itself.

In accordance with yet another embodiment, a tubular device is provided that includes at least one of an inflatable balloon, a working channel through which a biopsy device or other instrument may be advanced, and an ultrasound transducer. The tubular device may be advanced in an airway to a desired position, and then the balloon may be inflated, e.g., to compress adjacent lung tissue. Such compression may reduce the quantity of air in the tissue, facilitating improved transmission of ultrasound waves and, in turn, image quality. Optionally, the tubular device may include a plurality of balloons, e.g., on different distal branches, which may be advanced into adjacent airways in order to increase the compressive effects.

In accordance with still another embodiment, a sheath is provided that includes at least one of a working channel through which a biopsy device or other instrument may be advanced, and an ultrasound transducer. The sheath, and/or one of its constituent components has a directable terminal segment, whose purpose when actuated is to compress adjacent lung tissue in order to reduce air content and improve ultrasound image quality. The directable terminal portion may be linear, curvilinear, circular, or any other configuration to achieve the desired effect.

In accordance with an exemplary embodiment, a system is provided for performing a procedure within a patient's lung that includes an elongate tubular member including a proximal portion, a distal portion sized for introduction into a body lumen of a lung, and one or more lumens extending between the proximal and distal portions, thereby defining a longitudinal axis. An ultrasound imaging device is deployable from the distal portion for imaging tissue adjacent the body lumen, and an expandable member on the distal portion is configured to expand within the body lumen to isolate a region of the body lumen beyond the distal portion. A source of vacuum may be coupled to the proximal portion and communicating via a lumen of the tubular member with a port in the distal portion for aspirating fluid within region of the body lumen beyond the distal portion to collapse the body lumen around the imaging device.

In accordance with another exemplary embodiment, a method is provided for performing a procedure within a patient's lung that includes introducing a distal portion of an elongate tubular member into a body lumen of a lung; deploying an ultrasound imaging device from the distal portion within the body lumen; expanding an expandable member on the distal portion within the body lumen to isolate a region of the body lumen beyond the distal portion; aspirating fluid within the body lumen via the tubular member to at least partially collapse the body lumen around the imaging device; and activating the imaging device to identify a target tissue site adjacent the body lumen.

In accordance with still another embodiment, a method is provided for performing a procedure within a patient's lung that includes introducing a distal portion of an elongate tubular member into a body lumen of a lung; deploying an ultrasound imaging device from the distal portion within the body lumen; expanding an expandable member on the distal portion within the body lumen to isolate a region of the body lumen beyond the distal portion; delivering acoustic coupling fluid into the region of the body lumen beyond the expandable member via the tubular member; and activating the imaging device to identify a target tissue site adjacent the body lumen, the fluid enhancing acoustic coupling between the imaging device and tissue surrounding the body lumen.

In accordance with yet another embodiment, a method is provided for performing a procedure within a patient's lung that includes introducing a distal portion of an elongate tubular member into a body lumen of a lung; expanding an expandable member on the distal portion within the body lumen to distend a wall of the body lumen and compress lung tissue adjacent the wall to remove air within the tissue; and activating an imaging device within the balloon to acquire images of tissue adjacent the body lumen.

In accordance with another embodiment, a method is provided for performing a procedure within a patient's lung that includes introducing a distal portion of an elongate tubular member into a body lumen of a lung; manipulating the distal portion to position a first leg of the distal portion within a first branch of the body lumen and a second leg of the distal portion within a second branch adjacent the first branch; expanding expandable members on the first and second legs to compress lung tissue between the first and second branches; and activating an imaging device on the first leg to acquire images of the compressed lung tissue.

In accordance with still another embodiment, a method is provided for performing a procedure within a patient's lung that includes introducing a distal portion of an elongate tubular member into a body lumen of a lung; directing the distal portion to a deflected configuration to compress lung tissue adjacent the body lumen; and activating an imaging device on the distal portion to acquire images of the compressed lung tissue.

In accordance with yet another embodiment, a system is provided for accessing a body lumen within a patient's lung that includes a bronchoscope comprising a shaft sized for introduced into a patient's lung and a working channel; and an access sheath comprising a proximal portion, a distal portion, and one or more lumens extending therebetween, at least the distal portion being expandable from a contracted condition sized for introduction into the working channel of the bronchoscope to an expanded condition larger than the shaft.

In accordance with still another embodiment, a system is provided for performing a biopsy within a patient's lung that includes an elongate tubular member comprising a proximal portion, a distal portion sized for introduction into a body lumen of a lung, a central working lumen extending from the proximal portion to an outlet in the distal portion, thereby defining a longitudinal axis, and an accessory lumen disposed adjacent the working lumen; an ultrasound imaging device comprising a shaft slidably disposed within the accessory lumen, and an imaging element carried on a distal end of the shaft, the shaft movable axially between a retracted position wherein the imaging element is at least partially disposed within the working lumen and a deployed position wherein the imaging element is spaced apart from the distal portion of the tubular member; and a needle disposed within the working lumen of the tubular member and comprising a tip that is advanceable from the outlet of the working lumen when the imaging element is spaced apart from the distal portion, wherein the imaging element comprises a ramped proximal surface disposed adjacent to and aligned with the outlet of the working lumen in the deployed position for directing the tip of the needle laterally relative the longitudinal axis when the tip is advanced from the outlet.

Although discussed with particular applicability to accessing and/or performing procedures within a lung, it should be apparent to those skilled in the art that any of the devices, systems, and methods herein may be utilized in other body systems wherein a region of interest containing desired tissue, blood, or other body fluid or substance is within a certain proximity to a luminal structure, respectively, including but not limited to the cardiovascular system and constituent blood vessels, the gastrointestinal system and constituent digestive tract, the bile and pancreatic duct, the genitourinary system and constituent urethra, bladder, ureters, the nervous system and constituent blood vessels, and the ventricular system.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It will be appreciated that the exemplary apparatus shown in the drawings are not necessarily drawn to scale, with emphasis instead being placed on illustrating the various aspects and features of the illustrated embodiments.

FIGS. 1A-1D show an exemplary embodiment of a bronchoscope and a sheath that may be introduced over the bronchoscope to access a patient's lung, e.g., to introduce a biopsy instrument into the lung to perform a biopsy.

FIGS. 2A-2C show an exemplary embodiment of a bronchoscope and an expandable sheath that may be introduced through the bronchoscope to access a patient's lung, e.g., to introduce a biopsy instrument into the lung to perform a biopsy.

FIGS. 3A(1)-3C(2) are details showing exemplary embodiments of expandable sheaths that may be introduced through a bronchoscope, e.g., as shown in FIGS. 2A-2C.

FIGS. 4A and 4B are side views of a distal portion of an exemplary embodiment of an access sheath that includes lumens for receiving a biopsy instrument and an imaging device, e.g., to perform a biopsy at an area of interest.

FIGS. 5A and 5B are side views of a distal portion of another exemplary embodiment of an access sheath that includes lumens for receiving a biopsy instrument and an imaging device, e.g., to perform a biopsy at an area of interest.

FIGS. 6A and 6B are side views of a distal portion of an exemplary embodiment of a biopsy device including a hollow needle with internal threads.

FIG. 7 is a side view of a distal portion of yet another exemplary embodiment of an access sheath that includes a helical lumen for introducing a biopsy device.

FIG. 8 is a cross-sectional view of a body lumen showing the access sheath of FIG. 7 being used to introduce a biopsy device into an area of interest.

FIGS. 9A-9C are side views of an exemplary embodiment of another biopsy device.

FIGS. 10A-10D show the biopsy device of FIGS. 9A-9C being used to perform a biopsy.

FIGS. 11A and 11B are side views of an exemplary system for performing a biopsy that includes a biopsy device introduceable through an access sheath.

FIG. 12 is a side view of an exemplary embodiment of a fiducial marker.

FIGS. 13A-13C are side views of a system for performing a biopsy that includes a guide catheter and an imaging device and a needle introduceable through the guide catheter.

FIGS. 14A-14C are side views of another system for performing a biopsy that includes a guide catheter and an imaging device and a biopsy device introduceable through the guide catheter.

FIGS. 15A and 15B are cross-sectional views of a body lumen showing an ultrasound imaging device positioned within the body lumen for imaging surrounding tissue.

FIGS. 16A-16D are cross-sectional views of a lung showing exemplary methods for imaging a region of the lung that includes compressing aerated lung tissue to enhance imaging.

FIGS. 17A and 17B are cross-sectional views of a lung showing another exemplary method for imaging a region of the lung that includes compressing aerated lung tissue to enhance imaging.

FIGS. 18A and 18B are cross-sectional views of a body lumen showing yet another exemplary catheter and method for imaging a region adjacent the body lumen that includes changing the shape of a balloon carried on the catheter to modify a deployment angle of an instrument deployed from the catheter.

FIG. 19A is a side view of another embodiment of an access sheath or catheter carrying an imaging assembly including a transducer within a balloon and including an instrument lumen communicating with an outlet side port.

FIGS. 19B-19D are cross-sectional views of the sheath of FIG. 19A.

### DETAILED DESCRIPTION OF THE EXEMPLARY EMBODIMENTS

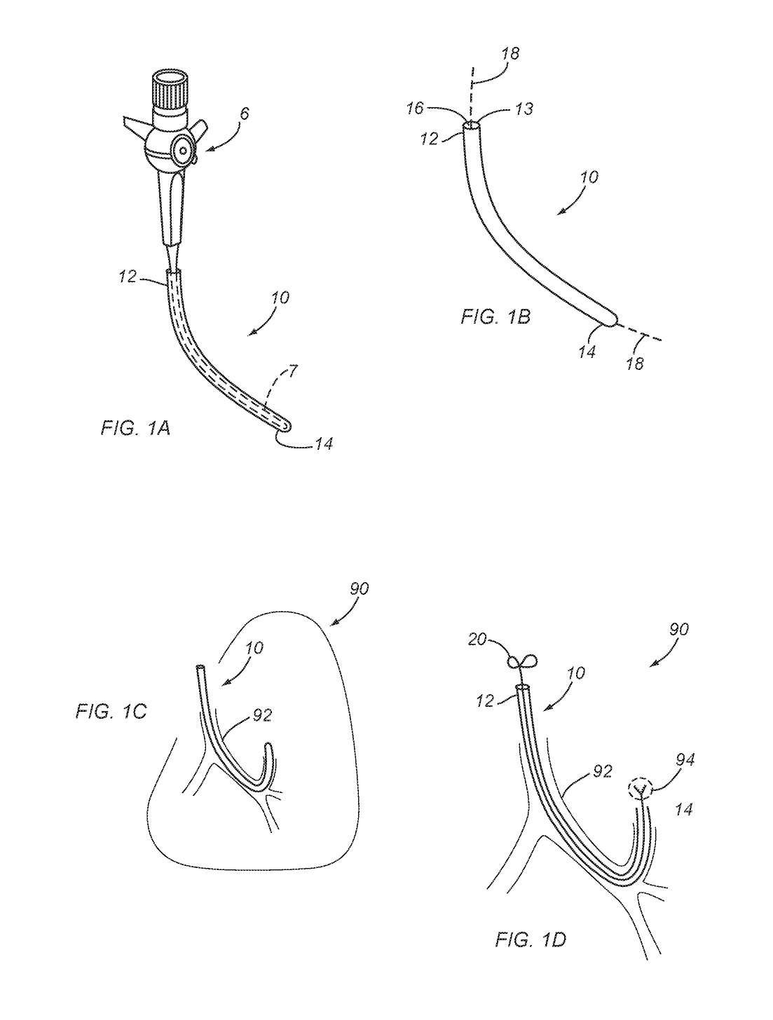

Turning to the drawings, FIGS. 1A-1D show an exemplary embodiment of an access sheath or catheter 10 for accessing a body lumen with a patient's body, e.g., an airway 92 within the patient's lung 90 (shown in FIGS. 1C and 1D), e.g., to image tissue, perform a biopsy, and/or other medical procedure. As described elsewhere herein, the sheath 10 may be part of a system for accessing and/or performing a procedure within the lung 90, e.g., including one or more other devices that may be introduced into and/or deployed from the sheath 10, such as a biopsy device or instrument 20 (shown in FIG. 1D) and/or an imaging device (not shown).

As best seen in FIG. 1B, the sheath 10 generally includes a proximal end or portion 12, a distal end or portion 14, and one or more lumens 16 extending therebetween, thereby defining a central longitudinal axis 18. For example, the sheath 10 may include one or more of an instrument or working lumen, an imaging device lumen, an aspiration or infusion lumen, and the like, as described further elsewhere herein.

Generally, the sheath 10 is formed from material that is sufficiently flexible to allow the sheath 10 to be placed over a shaft 7 of a bronchoscope 6, as shown in FIG. 1, which may be used to introduce the sheath 10 into the lung 90. Alternatively, the sheath 10 may have sufficient rigidity, e.g., a semi-rigid or rigid proximal portion and a flexible distal portion, to allow the sheath 10 to be introduced into the patient's body without the bronchoscope and/or other guide instrument.

As shown in FIG. 1B, optionally, the sheath 10 may include an elastic band 13 on the proximal end 12, which may be used to secure the sheath 10 temporarily to the bronchoscope 6 during delivery. Alternatively, the sheath 10 may include a handle or hub (not shown) on the proximal end 12, which may include one or more side ports (also not shown) communicating with respective lumens 16 in the sheath 10, as described elsewhere herein.

During use, the sheath 10 may be placed over the shaft 7 of the bronchoscope 6, as shown in FIG. 1A, and the sheath 10 and bronchoscope 6 may be introduced into the patient's body together, e.g., using conventional methods. For example, the bronchoscope 6 may include an imaging element on its distal end (not shown), which may provide direct visualization of the body lumen into which the bronchoscope 6 is introduced. Once the distal portion 14 of the sheath 10 is positioned at a desired location, e.g., within an airway 92 of the lung 90, as shown in FIGS. 1C and 1D, the bronchoscope 6 may be removed, leaving the sheath 10 to provide access into the airway 92 from outside the patient's body.

As shown in FIG. 1D, with the sheath 10 deployed within the airway 92 in the desired position, one or more instruments, e.g., a biopsy device 20, may be advanced through the sheath 10 to perform a procedure, e.g., to obtain a tissue sample from an area of interest 94 adjacent the airway 92. Further details and features of exemplary embodiments of sheaths, biopsy devices, imaging device, and systems including such devices are described further elsewhere herein.

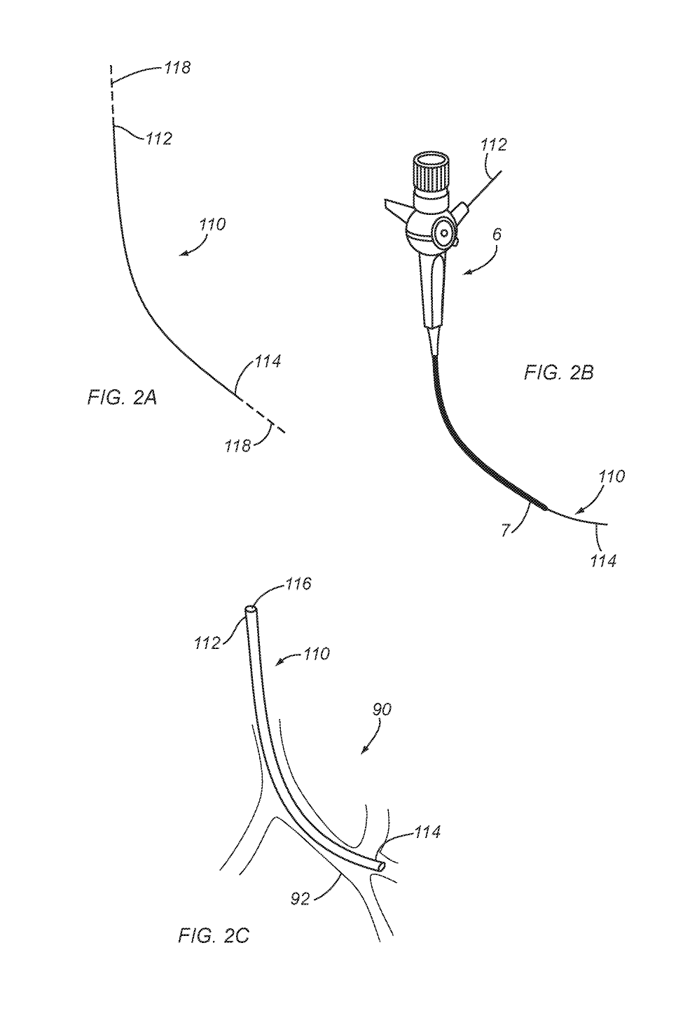

Turning to FIGS. 2A-2C, an alternate embodiment of an access sheath 110 is shown, which may be expandable from a contracted or collapsed configuration, e.g., shown in FIG. 2A, to an expanded configuration, e.g., as shown in FIG. 2C. For example, the sheath 110 may be configured to be folded, rolled, or otherwise compressed to reduce its cross-sectional dimension in order to allow at least the distal portion 114 of the sheath 110 to be introduced through a working channel of the bronchoscope 6. Once received in the bronchoscope 6, the distal portion 114 may extend from the shaft 7 of the bronchoscope 6 or may be disposed within the working channel, e.g., adjacent the distal end of the shaft 7.

For example, during use, the compressed sheath 110 may be loaded into the working channel of the bronchoscope 6 and then the bronchoscope 6 may be introduced into the patient's body, e.g., until the shaft 7 is positioned at a desired location in the airway 92. Alternatively, the bronchoscope 6 may be introduced first to position the shaft 7 within the airway 92 and then the distal portion 114 of the sheath 110 may be loaded into the working channel and advanced through the bronchoscope 6 until disposed within the airway 92, e.g., deployed from the shaft 7 or disposed within the shaft 7 adjacent the distal end.

The bronchoscope 6 may then be removed and the sheath 110 expanded within the airway 92, as shown in FIG. 2C. For example, as the bronchoscope 6 is removed, the distal portion 114 and then the remainder of the sheath 110 may resiliently return towards the expanded configuration. Optionally, in the expanded configuration, the sheath 110 may have a diameter or other maximum outer cross-section larger than the bronchoscope 6, which may maximize the available lumen space of the sheath 110 within the airway 92, e.g., for introducing one or more instruments via the sheath 110 thereafter.

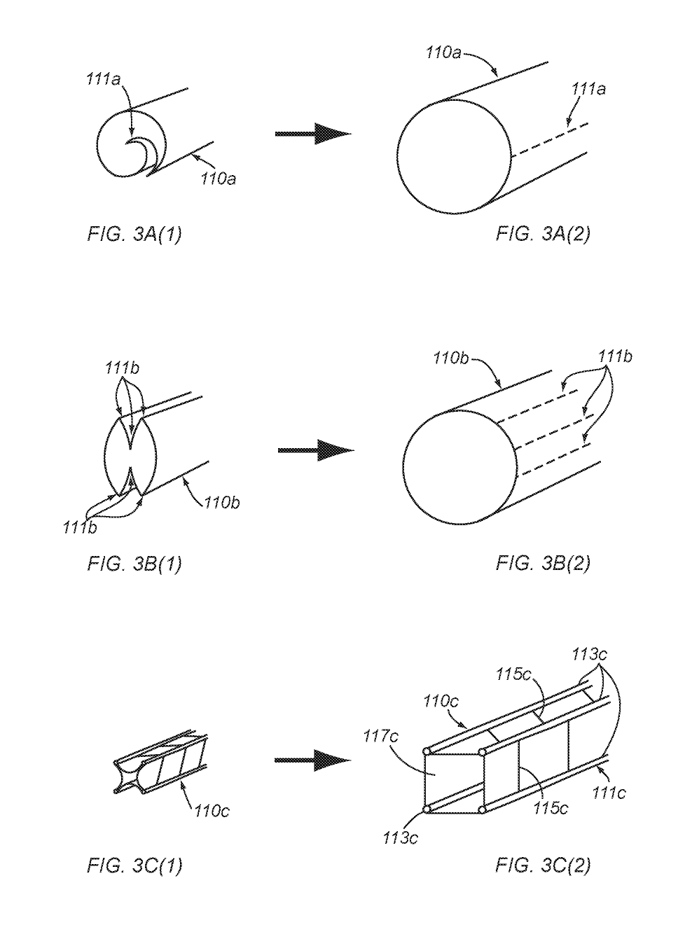

FIGS. 3A-3C show exemplary embodiments of expandable sheaths 110 that may be provided. In each of these embodiments, the sheath 110 may be directed to the contracted configuration (shown in FIGS. 3A(1), 3B(1), 3C(1)) yet resiliently biased to return to the expanded configuration (shown in FIGS. 3A(2), 3B(2), 3C(2) when no longer constrained, e.g., by the bronchoscope 6. Optionally, one or more clips, wires, or other constraints (not shown) may be provided to maintain the sheath 110 in the contracted configuration such that, upon removal of the constraint(s), the sheath 110 may become biased to expand. Alternatively, the bronchoscope 6 or other introduction device (not shown) may be sufficient to constrain the sheath 110 in the contracted configuration during delivery.

For example, FIGS. 3A(1) and 3A(2) show an exemplary sheath 110a in contracted and expanded configurations, respectively. The sheath 110a may be made from flexible material having some degree of shape memory. Along the longitudinal dimension of the sheath 110a, a crease 111a may be provided that facilitates folding the sheath 110a into itself, e.g., while maintaining a roughly circular shape in the contracted configuration and facilitating advancement through the working channel of the bronchoscope 6. Once no longer constrained by the working channel of the bronchoscope 6 (or by any constraints to maintain the compressed shape), the sheath 110a automatically deploys to its fully expanded configuration, e.g., with the residual crease 111a possibly remaining visible along its length, e.g., substantially parallel to the longitudinal axis 118a of the sheath 110a.

FIGS. 3B(1) and 3B(2) show another exemplary sheath 110b made from material having some degree of shape memory, having a total of six (6) creases 111b, which when folded, e.g., in alternating fashion, permit the sheath 110b to assume its contracted configuration that facilitates passage through the bronchoscopy 6 or other device designed to position the sheath 110b in the desired location. Once no longer constrained by the working channel of the bronchoscope 6, (or other constraint(s)), the sheath 110b resiliently expands towards the expanded configuration, thereby providing an increased cross-sectional dimension. The creases 111b may remain visible along the length of the sheath 110b, e.g., as shown in FIG. 3B(2).

FIGS. 3C(1) and 3C(2) show yet another exemplary sheath 110C, which includes a shape memory frame, e.g., formed from Nitinol or other elastic or superelastic material. In the embodiment shown, the frame 111c includes four longitudinal struts 113c (although optionally other numbers of struts may be provided), e.g., joined by several transverse connectors 115c, e.g., spaced apart from one another along the length of the sheath 110c.

The frame 111c may be entirely covered around the periphery of the sheath 110c with a membrane 117c, e.g., formed from relatively thin plastic or other polymer, fabric, and/or other flexible material. In the contracted configuration, the transverse connectors 115c may extend substantially parallel or near-parallel to the longitudinal struts 113c. Optionally, the sheath 110c may be rolled, folded, or otherwise compressed further along its longitudinal axis or otherwise modified or folded to further reduce the outer profile of the sheath 110c in the contracted configuration. Upon being deployed or released, the frame 111c may resiliently return towards its original expanded configuration, thereby opening the membrane 117c to provide one or more lumens extending along the sheath 110c.

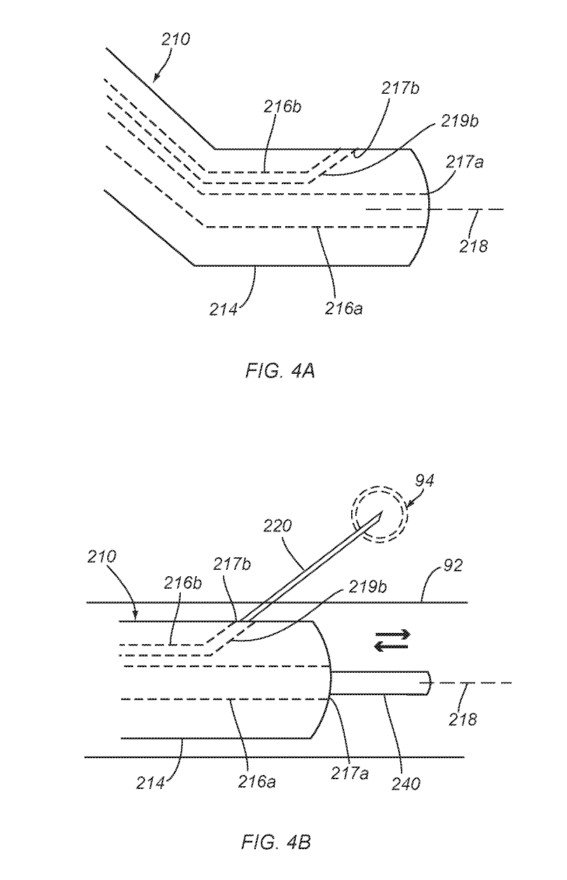

Turning to FIGS. 4A and 4B, a distal portion 214 of another exemplary embodiment of an access sheath or catheter 210 is shown that includes a pair of channels or lumens 216 that extend from the proximal end of the catheter 210 to respective ports or outlets 217 in the distal portion 214. Each lumen 216 may communicate with a hub on the proximal end (not shown), e.g., including a port allowing a corresponding device to be inserted into (and removed from) the lumen 216 and advanced, e.g., until deployed from the outlet 217. Alternatively, each lumen 216 may communicate to an actuator on a hub or handle on the proximal end (also not shown) such that the actuator may be used to deploy or retract the device disposed within the lumen 216.

The first or imaging lumen 216a is sized to receive an ultrasound probe or other real-time imaging instrument 240, while the second or working lumen 216b is sized to receive a needle or other biopsy device 220, e.g., as shown in FIG. 4B. As shown, the first lumen 216a may be disposed concentrically relative to the central longitudinal axis 218 of the distal portion 214, e.g., such that the outlet 217a is aligned with the central axis 218 and the imaging instrument 240 is substantially centered when deployed from the distal portion 214.

The second lumen 216b may extend adjacent to the first lumen 216a, e.g., offset from the central axis 218, and the outlet 217b may be located on a side wall of the distal portion 214. The second lumen 216b may include a ramped surface 219b adjacent the outlet 217b, e.g., such that the second lumen 216b changes direction at a specific angle relative to the central axis 218. Thus, a biopsy device 220 deployed from the outlet 217b may extend laterally relative to the central axis 218 and the distal portion 214. The catheter 210 may be manufactured with various angulations to achieve the desired directionality of the biopsy device 220 when deployed.

Similar to other embodiments herein, the catheter 210 may be substantially rigid, semi-rigid, or flexible, e.g., having a variable rigidity along its length, e.g., being more rigid at the proximal portion to facilitate advancement, rotation, and/or other manipulation of the distal portion 214 from outside the patient's body, and being flexible at the distal portion 214 to facilitate introduction through tortuous anatomy. Optionally, the distal portion 214 may be biased to a predetermined shape, e.g., a simple curve or other curvilinear shape, yet may be resiliently deflected towards other shapes, e.g., to adopt the shape of a bronchoscope or other guide instrument used to deliver the catheter 210 and/or to facilitate introduction of the catheter 210 independent of a guide instrument, if desired.

With reference to FIG. 4B, during use, the distal portion 214 may be positioned within an airway or other body lumen 92, e.g., in conjunction with a bronchoscope or other guide instrument (not shown), similar to other embodiments herein. Once positioned at a desired location, an imaging device 240, e.g., a radial EBUS probe, may be inserted into the first lumen 216a and deployed from the outlet 217a into the body lumen 92. The imaging device 240 may be activated and manipulated, e.g., axially relative to the distal portion 214, to best visualize the area of interest. Optionally, the imaging device 240 may be rotatable relative to the distal portion 214, e.g., if the imaging device 240 is directional or the entire distal portion 214 may be rotated to direct the imaging device 240.

Once a target site 94 is identified, e.g., for biopsy, a biopsy device, e.g., biopsy needle 220, may be deployed from the outlet 217b of the second lumen 216b. For example, using real-time images from the imaging device 240, the distal portion 214 may be rotated and/or directed axially to align the target site 94 with the outlet 217b of the second lumen 216b. The biopsy needle 220 may be introduced into the second lumen 216b, e.g., via a port on the proximal end (not shown), before or after positioning the distal portion 214. By moving the catheter 210 and biopsy device 220 axially relative to each other and relative to the airway 92 and area of interest 94, the needle 220 may be situated such that the needle 220 enters the area of interest (407) when advanced from the outlet 217b through the wall of the airway 92. Similarly, the distal portion 214 of the catheter 210 may be rotated about the central axis 218 to further direct the needle 220 towards the area of interest 94.

Optionally, the imaging device 240 may include a processor and display (not shown), e.g., a radial EBUS system, and the processor may analyze the images acquired by the imaging device 240 to present one or more markers on the display to indicate the location, direction, and/or orientation of the needle 220 with respect to the area of interest 94. Thus, the catheter 210 may be used to perform a biopsy or otherwise acquire a sample of tissue from the area of interest 94 using the needle 220, which may then be analyzed to aid in diagnosis and/or treatment of the patient.

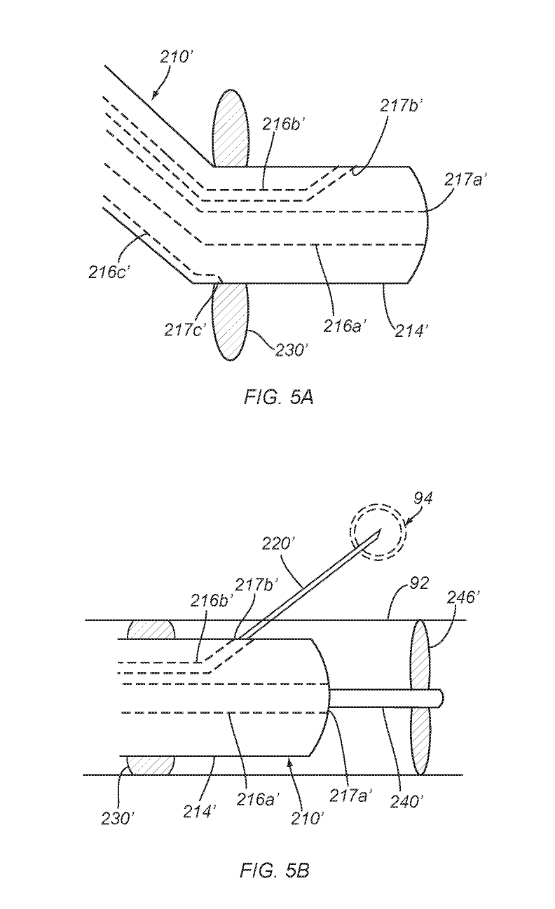

Optionally, one or more balloons or other expandable members may be provided on one or more components of the system to facilitate performing a biopsy or other procedure. For example, FIGS. 5A and 5B show an alternative embodiment of a catheter 210′ generally similar to the catheter 210 shown in FIGS. 4A and 4B. For example, the catheter 210′ generally includes a proximal portion (not shown), a distal portion 214′ sized for introduction into a patient's body, and a plurality of channels or lumens 216′ that extend from the proximal end of the catheter 210′ to respective ports or outlets 217′ in the distal portion 214.′ The first or imaging lumen 216a′ is sized to receive an ultrasound probe or other real-time imaging instrument 240,′ while the second or working lumen 216b′ is sized to receive a needle or other biopsy device 220,′ e.g., as shown in FIG. 5B.

In addition, the catheter 210′ includes a balloon or other expandable member 230′ on the distal portion 214,′ e.g., proximal to the outlet 217b′ of the working lumen 216b.′ The catheter 210′ may include an inflation lumen 216c′ extending from a port on the proximal end (not shown) to a side port 217c′ communicating with an interior of the balloon 230,′ as shown in FIG. 5A, e.g., for inflating and/or collapsing the balloon 230.′ The balloon 230′ may include an annular membrane and the like, e.g., formed from compliant or semi-compliant material, and attached at its ends to the distal portion 214,′ e.g., by bonding with adhesive, sonic welding, fusing, and the like.

The balloon 230′ may be expanded within an airway 92 or other body lumen, e.g., as shown in FIG. 5B, to substantially fix the distal portion 214′ within the airway 92. For example, the balloon 230′ may be expanded after positioning the distal portion 214′ to orient the outlet 217b′ towards the area of interest 92, e.g., based on images from the imaging device 240,′ before deploying the needle 220′ into the area of interest 94 to prevent migration of the distal portion 214′ during deployment, e.g., similar to the procedures described with reference to the catheter 210 shown in FIGS. 4A and 4B.

In addition or alternatively, the balloon 230′ may substantially isolate the region of the airway 92 beyond the balloon 230′ and/or beyond the distal portion 214,′ e.g., from the region of the airway 92 proximal to the balloon 214.′ For example, with the balloon 214′ inflated, one or more fluids, e.g., water, saline solution, and/or other ultrasound conductive fluid, may be injected into the region beyond the balloon 214′ to enhance acoustically coupling the imaging device 240′ to tissue surrounding the airway 92. Optionally, one or more other compounds, e.g., sclerosing material, may be injected into the region in addition to or instead of ultrasound conductive fluid. Such fluid(s) may be injected through one of the first and second lumens 217′ or through a dedicated infusion lumen having one or more outlet ports (not shown) on the distal portion 214.′

In addition or alternatively, fluid within the region of the airway 92 beyond the balloon 230′ may be aspirated using the catheter 214.′ For example, with the balloon 230′ substantially isolating the region, a suction force may be applied to the region of the airway 92, e.g., using a source of vacuum (not shown) coupled to the proximal end of the catheter 214′ and one of the lumens 216,′ to at least partially collapse the airway 92 and/or the surrounding tissue, which may enhance acoustically coupling the imaging device 240′ to the surrounding tissue.

Optionally, as shown in FIG. 5B, the imaging device 240′ may also include a balloon or other expandable member 246,′ which may be expanded when the imaging device 240′ is deployed from the distal portion 214.′ The balloon 246′ may be used to substantially fix the imaging device 240′ and/or substantially center the imaging device 240′ relative to the airway 92, e.g., while allowing the catheter 214′ to be manipulated further before deploying the needle device 220′ into the area of interest 94.

It should be noted that these exemplary embodiments of the catheter 210, 210′ and associated devices may be introduced into an airway 92 to access adjacent parenchyma. However, such catheters may also be used to access an area of interest in any area of the body that is adjacent to a lumen, for example, a lumen within the gastrointestinal system, biliary system, pancreas, and cardiovascular system.

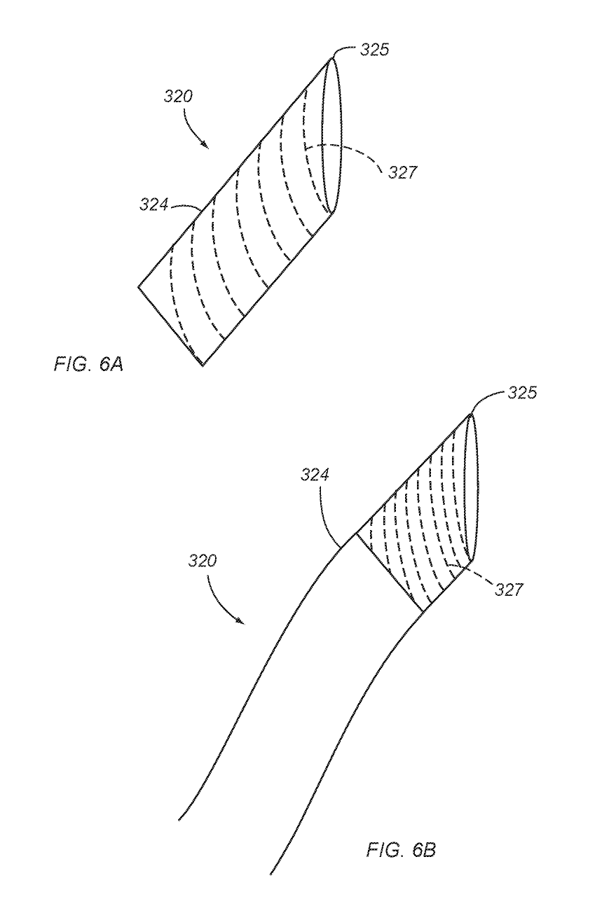

Turning to FIGS. 6A and 6B, an exemplary biopsy needle device 320 is shown that may be used in cooperation with any of the embodiments of catheters and access sheaths described elsewhere herein. Generally, the needle device 320 includes a catheter or other elongate member including a proximal end (not shown) and a distal portion 324 sized for introduction through a working or instrument lumen of an access sheath (not shown). The distal portion 324 may carry a needle 325 that terminates in a pointed, beveled, and/or other sharpened tip. The external surface of the distal portion 324 of the needle 325 may be smooth. However, the interior region of the needle 325 is hollow and the interior surface includes one or more helical threads 327. The thread(s) 327 may be configured such that, when a simultaneous forward and rotational force is applied to the distal portion 324, the needle 325 may be advanced into an area of interest (not shown), thereby directing tissue from the area of interest into the hollow region engaging the thread(s) 327. The needle 325 may then be withdrawn, e.g., by directing the distal portion 324 by pulling proximally from the proximal end without rotation, thereby separating the tissue within the hollow region of the needle 325 from the area of interest with the internal thread(s) 327 retaining the tissue to remain in situ during the removal process.

The needle 325 may be relatively short compared to the catheter, e.g., having a length between about two and fifty millimeters, which may facilitate advancing the needle device 320 through the access sheath, even if oriented in a sharply curving shape through body lumens of the patient's body, without substantial risk of skiving, catching, or otherwise damaging the lumen wall of the access sheath. Optionally, multiple needle devices may be inserted into the access sheath, used to acquire tissue sample, and removed sequentially, e.g., to obtain multiple samples using the same access sheath at the same or different locations.

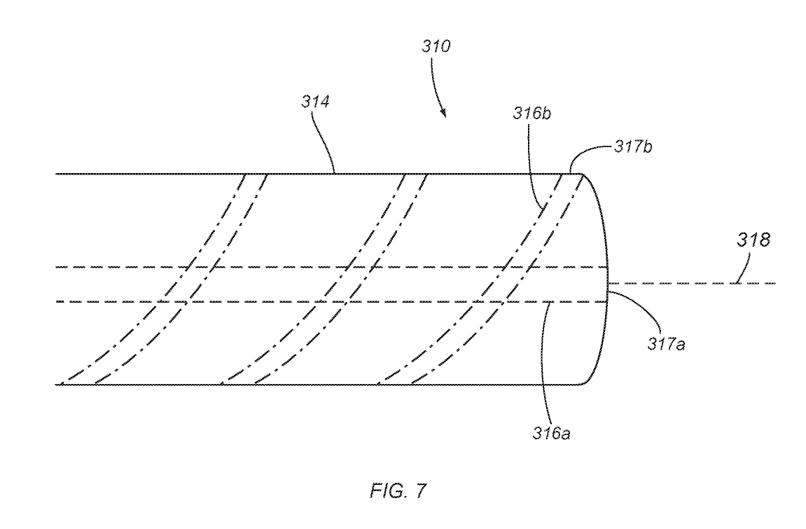

Turning to FIG. 7, another exemplary embodiment of a catheter 310 is shown that generally includes a proximal end (not shown), a distal portion 314 sized for introduction into a body lumen, and a pair of lumens 216 extending therebetween, similar to other embodiments herein. For example, a first lumen 316a may extend along the distal portion 314 to an outlet 317a generally aligned with a central longitudinal axis 318 of the catheter 310, e.g., for deploying a real-time imaging instrument such as endobronchial ultrasound or other imaging device (not shown). A second lumen 316b is also provided within the distal portion 314 that may be located near the outer circumference of the catheter 310 and terminates at an outlet 317b in a side wall of the distal portion 314, e.g., for receiving a biopsy device (not shown). At least a portion of the second lumen 316b has a helical shape, e.g., within the distal portion 314 and may transition to an axial orientation proximal to the distal portion 314. In this configuration, a needle or other biopsy device introduced through the second lumen 316b may be deployed laterally relative to the central axis 318 and may also extend diagonally.

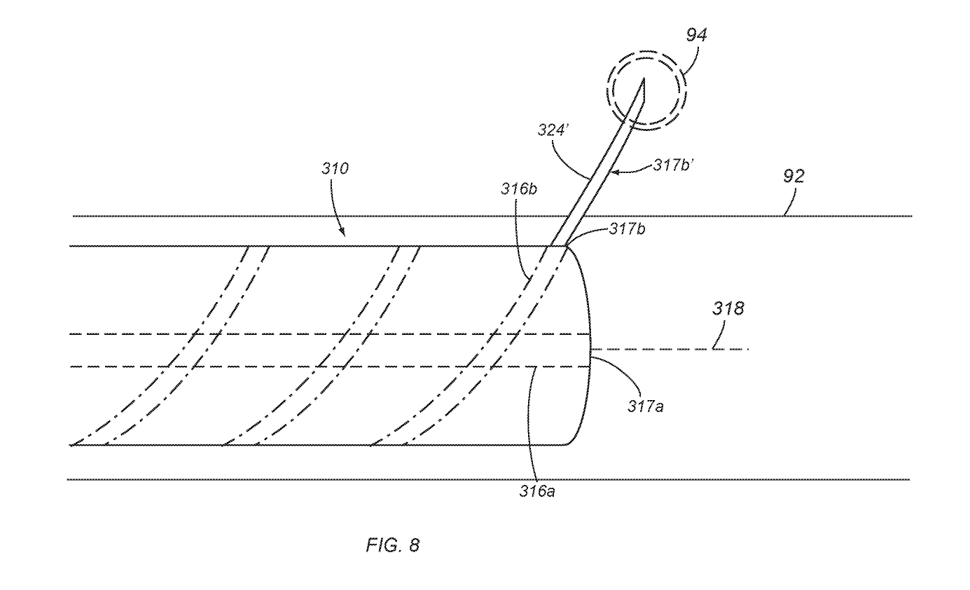

During use, as shown in FIG. 8, the distal portion 314 may be positioned within an airway or other body lumen 92 adjacent to an area of interest 94, similar to other embodiments herein. A radial EBUS probe or other imaging device 340 may be deployed from the first lumen into the airway 92, e.g., to obtain images of the tissue surrounding the airway 92. A biopsy needle device 320′ may be introduced into the second lumen 316b, which may include a distal portion 324′ having a curvature that matches that of the helical region of the second lumen 316b. The distal portion 324′ may be coupled to a flexible catheter or other elongate member (not shown) that extends through the catheter 310 to the proximal end. The needle device 320′ may thus be advanced under real-time visualization of the EBUS probe 340 into the area of interest 94. Optionally, the needle device 320′ and/or imaging device 340 may include features similar to other embodiments herein.

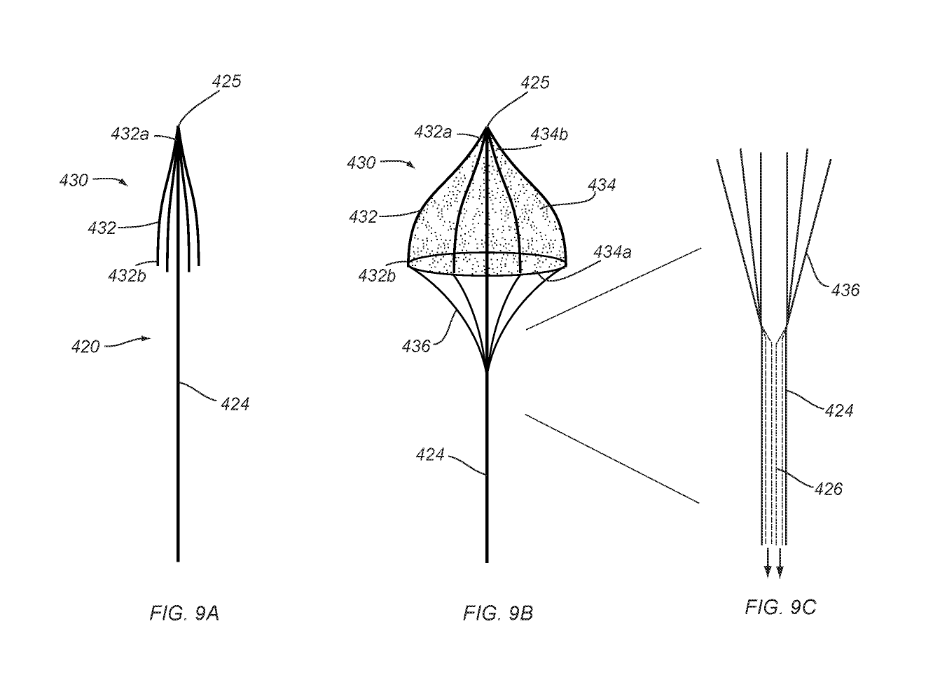

Turning to FIGS. 9A-9C, another exemplary embodiment of a biopsy device 420 is shown that may be delivered using any of the access sheaths, catheters, and systems herein to perform a biopsy, e.g., within a patient's lung or other tissue. Generally, the biopsy device 420 includes a catheter or other elongate shaft 422 including a proximal portion (not shown), a distal portion 424 sized for introduction through a lumen of an access sheath, and an expandable capture structure 430 carried on the distal portion 424, which is movable between a compressed configuration (shown in FIG. 9A) intended to advance the distal portion 424 at least partially into an area of interest within a tissue structure and a deployed configuration (shown in FIG. 9B) intended to capture a tissue sample from the area of interest. The distal portion 424 may terminate in a pointed, beveled, or other sharpened tip 425 to facilitate puncturing through tissue.

As shown in FIGS. 9A and 9B, the capture structure 430 includes a plurality of struts 432 including first ends 432a attached to or otherwise coupled to the distal portion 424 and second free ends 432b, and a sheeting material or membrane 434 carried by the struts 432. Although four struts 432 are shown, the capture structure 430 may include two, three, or more struts, as desired. The struts 432 may be formed from elastic, superelastic, and/or shape memory material, e.g., Nitinol, such that the free ends 432b of the struts 432 are biased to the deployed configuration shown in FIG. 9B, yet resiliently compressed inwardly towards the distal portion 424 to the compressed configuration.

As shown in FIG. 9B, with the struts 432 in the deployed configuration, the membrane 434 may be opened to define an open proximal end 434a and a closed distal end 434b adjacent the distal tip 425. The membrane may be formed from a variety of materials, e.g., biocompatible plastic, fabric, and the like, which may be nonporous or porous. In an alternative embodiment, if sufficient numbers of struts are provided, the membrane may be omitted and/or a mesh or other porous structure may be carried by the struts to capture tissue within the capture structure 430.

Optionally, the capture structure 430 may include one or more actuator elements, e.g., to facilitate collapsing the capture structure 430 after capturing a tissue sample. For example, as shown in FIGS. 9B and 9C, a plurality of flexible or rigid wires, strings, or other filaments 436 may be coupled to the free ends 432b of the struts 432 that enter one or more openings 438 in the distal portion 424 and extend through a lumen 426 to the proximal end of the biopsy device 420.

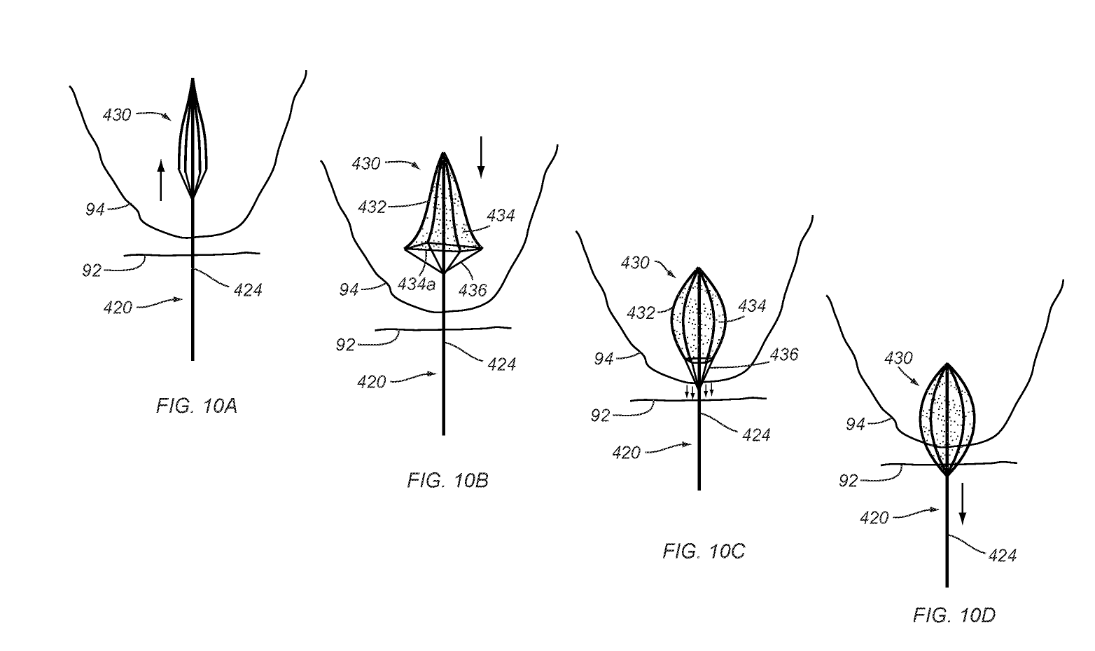

Turning to FIGS. 10A-10D, an exemplary method is shown for using the biopsy device 420 of FIGS. 9A-9C, e.g., to obtain a tissue sample within lung tissue 94 adjacent an airway 92. The distal portion 424 of the biopsy device 420 may be loaded into an access sheath, catheter, or other delivery device (not shown), e.g., within the biopsy lumen 216b of the catheter 210 shown in FIGS. 4A-4B, with the capture structure 430 in the compressed configuration shown in FIG. 10A. After positioning the delivery device within the airway adjacent an area of interest, e.g., using ultrasound imaging and/or other guidance (e.g., similar to the methods used to position and/or orient the outlet 217b of the catheter 210 shown in FIGS. 4A-4B towards the area of interest 94), the distal portion 424 may be deployed from the delivery device, e.g., laterally into the area of interest 94, by advancing the proximal end of the biopsy device 420 from outside the patient's body. The sharpened distal tip 425 may facilitate easy advancement into the tissue of the area of interest 94 with the capture structure 430 remaining substantially in the compressed configuration, as shown in FIG. 10A.

Turning to FIG. 10B, once the capture structure 430 is fully inserted into the tissue 94, the capture structure 430 may be deployed by partially retracting the capture structure 430 within the area of interest 94. This action may cause the free ends 432b of the struts 432 to engage tissue and move outwardly relative to the distal portion 424, thereby expanding the membrane 434 as shown. Optionally, the expansion and/or other manipulation of the capture structure 430 may be monitored, e.g., using an imaging device within the airway 92 and/or using external imaging, e.g., fluoroscopy, ultrasound, MRI, and the like, to ensure that the membrane 434 expands and target tissue is located between the open proximal end 434a and the airway 92.

As shown in FIG. 10C, the capture structure 430 may then be closed around the captured tissue, e.g., by pulling the control filaments 436 to draw the free ends 432b of the struts 432 inwardly and close the membrane 434 around the captured tissue. As shown in FIG. 10D, the closed capture structure 430 may then be retracted out of the area of interest 94, e.g., back into the lumen of the delivery device and removed from the patient's body.

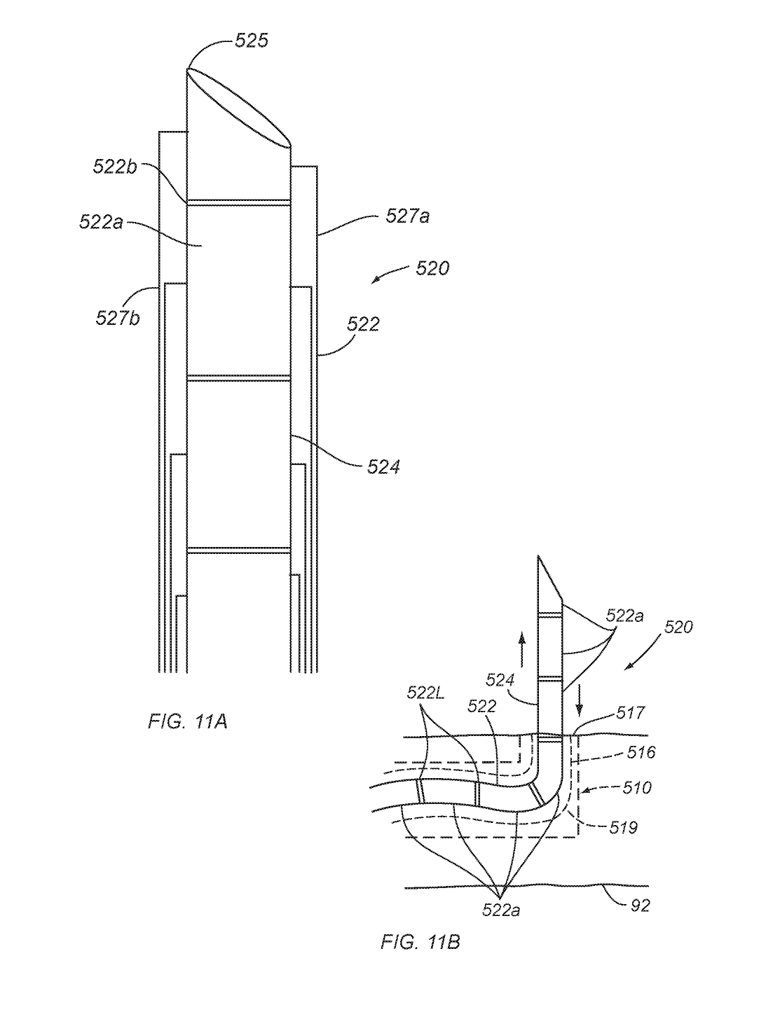

Turning to FIGS. 11A and 11B, another exemplary embodiment of a biopsy device 520 is shown that includes a shaft 522 including a proximal end (not shown) and a distal portion 524 that terminates in a needle tip 525. The shaft 522 may be formed from a plurality of segments coupled sequentially to one another such that a rigidity of the shaft 522 may be selectively modified, e.g., using piezoelectric principles. As shown, the shaft 522 includes several segments 522a made from piezoelectric material, with adjacent segments separated by layers of insulating material 522b. A plurality of wires or other conductive elements 527, e.g., positive wires 527a and negative wires 527b may be coupled to each segment, which may be used to selectively apply current to respective segments to modify their material properties when desired.

As shown in FIG. 11B, during use, an access sheath, catheter, or other delivery device 510 (shown in phantom) may be introduced into an airway 92, similar to other embodiments herein, that includes a working channel or lumen 516 including a ramped surface 519, e.g., defining a right angle, adjacent an outlet 517 through which the biopsy device 520 may pass. With the segments 522a disposed within the working lumen 516, no current is applied, e.g., such that the segments 522a are in a relatively flexible or semi-rigid state relative to one another, which may facilitate advancing the distal portion 524 through the delivery device (particularly if the delivery device has several bends from being introduced through tortuous anatomy). As each segment 522a advances around the ramped surface 519, electric current may be applied to the segments 522a, thereby causing the segments 522a to become substantially rigid relative to one another. Thus, the distal portion 524 of the biopsy device 520 that exits the outlet 517 may adopt a substantially rigid state, allowing the needle tip 525 to be penetrated into tissue and/or otherwise directed into an area of interest without risk of buckling, e.g., to obtain a tissue sample within a recess within the needle tip 525.

When the distal portion 524 is retracted back into the working lumen 516, electric current may be removed from the segments 522a as they enter the lumen 516, thereby returning the segments 522a to a flexible or semi-rigid state. A variety of mechanisms and systems may be used to determine the sequence and timing of applying electric current to each segment. For example, a controller, e.g., in a handle in the proximal end (not shown) of the biopsy device 520 or otherwise disposed externally to the patient, may be coupled to the wires 527 to selectively apply current to desired segments 522 automatically, e.g., in response to one or more position sensors or other feedback elements in the delivery device 510, e.g., adjacent the outlet 517 or ramped surface 519, that indicate when individual segments exit or enter the outlet 517 or are adjacent the ramped surface 519. Alternatively, with relative lengths known, the controller may activate the segments based on the extent and/or time that the biopsy device is inserted into the delivery device. In a further alternative, the user may manually activate desired segments to provide desired rigidity to the shaft 522.

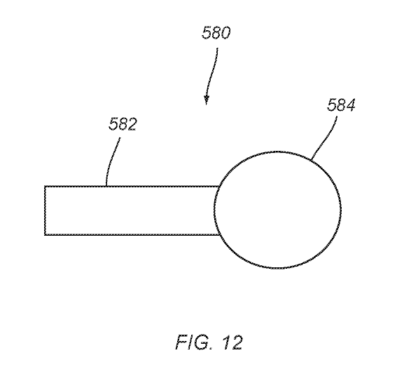

Turning to FIG. 12, an exemplary embodiment of a fiducial marker 580 is shown that may be used in conjunction with any of the systems and methods herein. Generally, the fiducial marker 580 includes a rigid component 582, e.g., a shaft configured to be palpated through a desired portion of tissue, and a light source 584 coupled to the shaft 582. In one embodiment, the shaft 582 may include a biocompatible metal segment and the light source 584 may be an LED light source configured to emit visible light. The light source 582 may be powered by a battery or other power source (not shown) within the marker 580, or alternately by an external power source, which may deliver power to the marker 580 wirelessly, e.g., using an external induction device (not shown), or by an external wired power source. For example, the light source 584 may be selectively activated by placing an external electromagnetic energy source adjacent to a tissue region within which the marker 580 has been implanted, e.g., using conventional devices and methods.

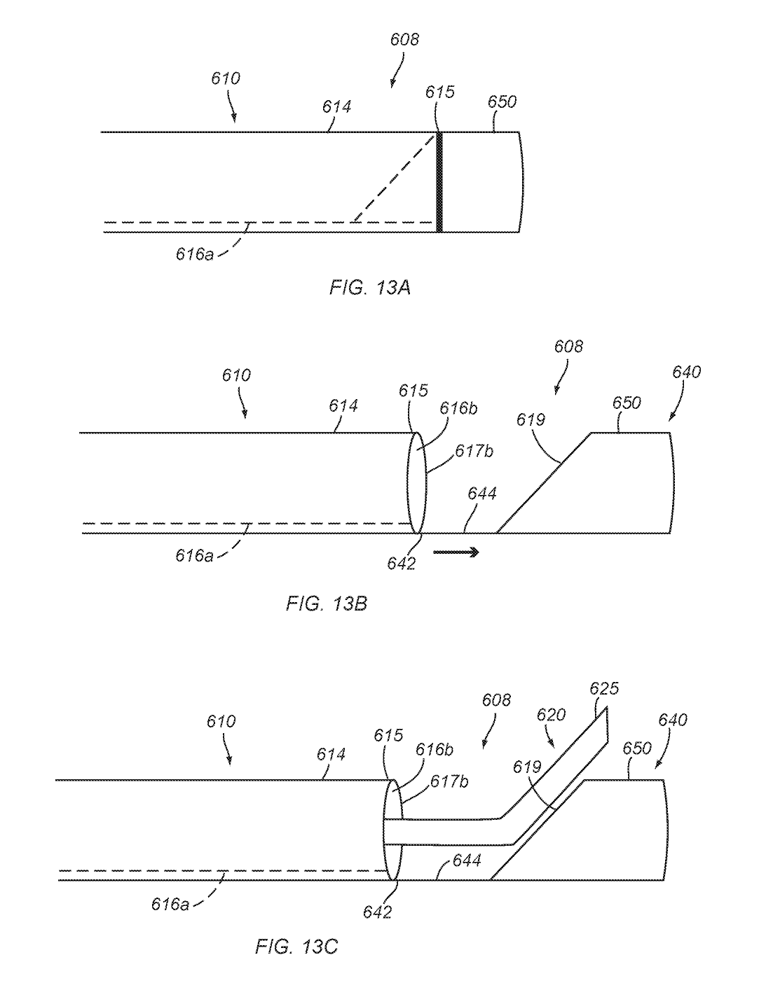

Turning to FIGS. 13A-13C, another exemplary embodiment of a system 608 for imaging and/or performing a biopsy within a patient's lung or other region is shown that includes an access sheath or catheter 610, a biopsy device 620, and an imaging device 640, which may be constructed similar to other embodiments herein. Generally, the catheter 610 includes a proximal end or portion (not shown), a distal end or portion 614, and one or more channels or lumens 616 extending from the proximal end to the distal portion 614. For example, the catheter 610 may include a working lumen 616b communicating with an outlet 617b sized to receive a needle or other biopsy device 620, e.g., as shown in FIG. 13C, which may be similar to any of the biopsy devices described elsewhere herein.

The imaging device 640 generally includes a shaft 642 including a proximal end, e.g., within the proximal end of the catheter 610 (not shown), and a distal end 644 carrying an imaging element 650, e.g., including one or more piezoelectric transducers or other ultrasound imaging elements (not shown) within a housing. The shaft 642 may include one or more lumens or other regions that contain one or more wires or other conductive elements and/or components (also not shown) needed to operate the imaging element 650.

The shaft 642 may be slidably disposed within an imaging lumen 616a, which may be relatively small compared to the working lumen 616b, such that the imaging element 650 may be movable between a proximal or retracted position (shown in FIG. 13A) and a distal or deployed position (shown in FIGS. 13B and 13C). In the retracted position, the imaging element 650 may be at least partially received within the working lumen 616a (e.g., as shown in FIG. 13A), e.g., to provide a transition for introduction of the distal portion 614, while in the deployed position, the imaging element 650 may be spaced apart from a distal tip 615, e.g., to provide a ramped surface 619 adjacent the outlet 617b of the working lumen 616b.

During use, the distal portion 614 of the catheter 610 may be introduced into a patient's body, e.g., into an airway in a lung (not shown), similar to other embodiments herein, but with the imaging element 650 in the retracted position shown in FIG. 13A. Optionally, the imaging device 640 may include a rounded or other substantially atraumatic tip (not shown), which may facilitate introduction of the catheter 610. Once positioned within a desired airway or other body lumen, the imaging element 650 may be deployed, as shown in FIG. 13B, and activated as desired, e.g., to provide real-time imaging to facilitate positioning the imaging element 650 and/or the ramped surface 619 adjacent an area of interest (not shown). Thus, the distal portion 614 of the catheter 610 and/or imaging element 650 may be manipulated axially and/or rotated as desired, e.g., to image an area of interest and/or direct the ramped surface 619 towards the area of interest.

Once positioned as desired, a biopsy device, e.g., a needle device 620, may be deployed from the working lumen 616b out the outlet 617b. As shown in FIG. 13C, as the tip 625 of the needle device 620 exits the outlet 617b, the tip 625 may slidably contact the ramped surface 619, thereby deflecting the tip 625 and directed the needle device 620 laterally relative to the catheter 610. For example, the needle device 620 may be introduced into the catheter 610, advanced from the outlet 617b into an area of interest adjacent the airway to perform a biopsy, similar to other embodiments herein.

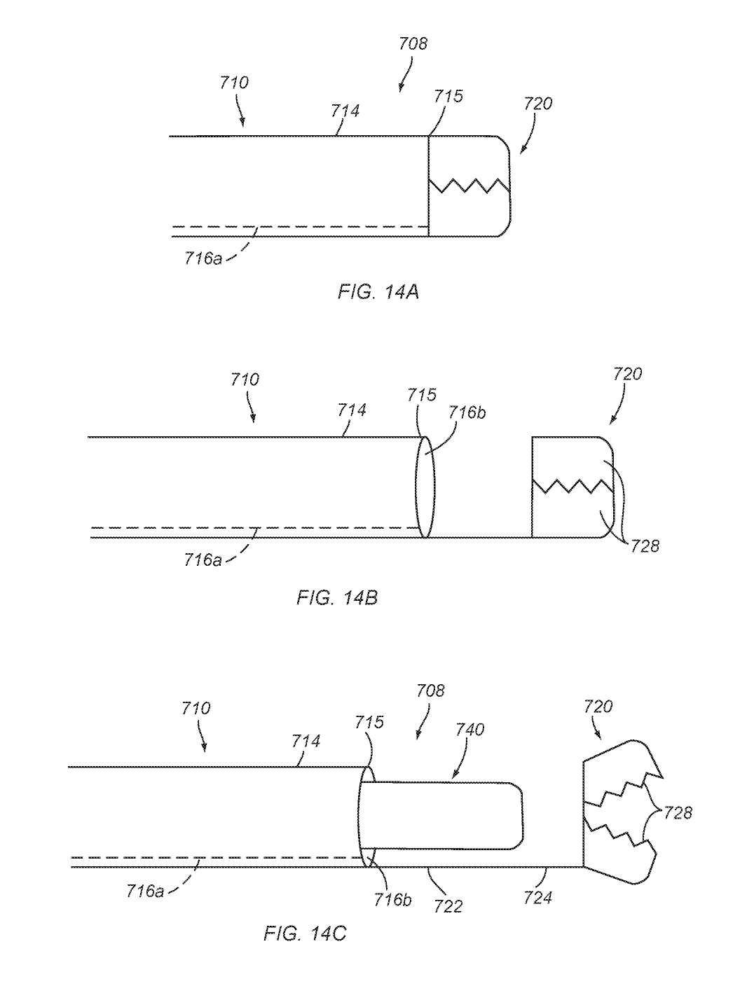

Turning to FIGS. 14A-14C, still another embodiment of a system 708 for imaging and/or performing a biopsy within a patient's lung or other region is shown that includes an access sheath or catheter 710, a biopsy device 720, and an imaging device 740, which may be constructed similar to other embodiments herein. Generally, the catheter 710 includes a proximal end or portion (not shown), a distal end or portion 714, and one or more channels or lumens 716 extending from the proximal end to the distal portion 714. For example, the catheter 710 may include a working lumen 716b sized to receive the imaging device 740, which may be generally aligned with a central axis of the catheter 710 e.g., as shown in FIG. 14C.

The biopsy device 720 generally includes a shaft 722 including a proximal end, e.g., within the proximal end of the catheter 710 (not shown), and a distal end 724 carrying a biopsy element, e.g., a set of forceps 728. The shaft 722 may include one or more lumens or other regions that contain one or more rods, wires or other actuator elements and/or components (also not shown) for operating the forceps 728.

The shaft 722 may be slidably disposed within an accessory lumen 716a, which may be relatively small compared to the working lumen 716b, such that the forceps 728 may be movable between a proximal or retracted position (shown in FIG. 14A) and a distal or deployed position (shown in FIGS. 14B and 14C). In the retracted position, the forceps 728 may be at least partially received within the working lumen 716b (e.g., as shown in FIG. 13A), e.g., to provide a transition for introduction of the distal portion 714, while in the deployed position, the forceps 728 may be spaced apart from a distal tip 715.

During use, the distal portion 714 of the catheter 710 may be introduced into a patient's body, e.g., into an airway in a lung (not shown), similar to other embodiments herein, but with the forceps 728 in the retracted position shown in FIG. 14A. Optionally, the forceps 728 may include rounded or other substantially atraumatic tips (not shown), which may facilitate introduction of the catheter 710. Once positioned within a desired airway or other body lumen, the forceps 728 may be deployed, as shown in FIG. 14B.

The imaging device 740 may then be deployed from the working lumen 716b, e.g., to provide real-time imaging to facilitate positioning the forceps 728 during a procedure. For example, one or more of the distal portion 714 of the catheter 710, the forceps 728, and/or imaging device 740 may be manipulated axially and/or rotated as desired, e.g., individually or together, to image an area of interest and/or direct the forceps 728 to an area of interest adjacent the airway to perform a biopsy, similar to other embodiments herein.

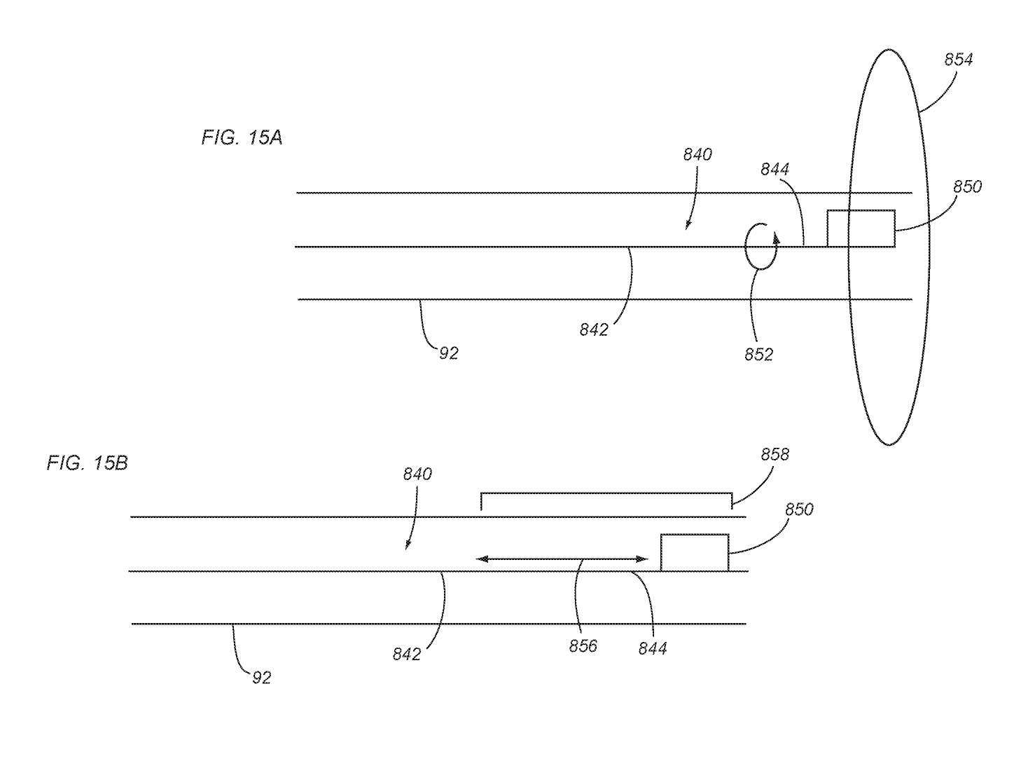

Turning to FIGS. 15A and 15B, an exemplary embodiment of an ultrasound imaging device 840 is shown, which may be used in conjunction with any of the systems and methods herein, e.g., for imaging within an airway or other body lumen 92. Generally, the imaging device 840 includes a shaft or other elongate member 842 including a proximal end (not shown), and a distal end 844 carrying one or more imaging elements, e.g., an ultrasound transducer 850 oriented to acquire images laterally, e.g., substantially perpendicular to a longitudinal axis of the shaft 842. The shaft 842 may be sufficiently flexible to facilitate introduction into the airway 92, e.g., via an access sheath, catheter, or other delivery device (not shown), and have sufficient column and torsional strength such that torsional and linear forces (e.g., as represented by arrows 852 and 856) may be transmitted to the distal end 844 from the proximal end.

The imaging device 840 may be coupled to a controller, e.g., at the proximal end (not shown), which may be used to activate the transducer 850 in one of two modes of operation. For example, in a first mode, a rotational force 852 may be applied to the shaft 842 to rotate the transducer 850 and acquire radial ultrasound images, e.g., within an image slice plane 854 shown in FIG. 15A. In a second mode, the rotational force 852 may be stopped and an axial force 856 may be applied to direct the shaft 842 proximally and distally, e.g., to move the transducer 850 back and forth axially within the airway 92 and acquire linear ultrasound images, e.g., along a desired two-dimensional linear axis 858 shown in FIG. 15B.

In an exemplary method, a user may manipulate the imaging device 840 while the transducer 850 is rotating to localize a region of interest, e.g., to acquire annular image slices of a tissue region adjacent the airway 92. Once the region is identified, the transducer 850 may be used to acquire images in the second, linear mode, e.g., to acquire linear or axial wedge-shaped images more suitable for real-time visualization of a biopsy device (not shown) being directed into the region.

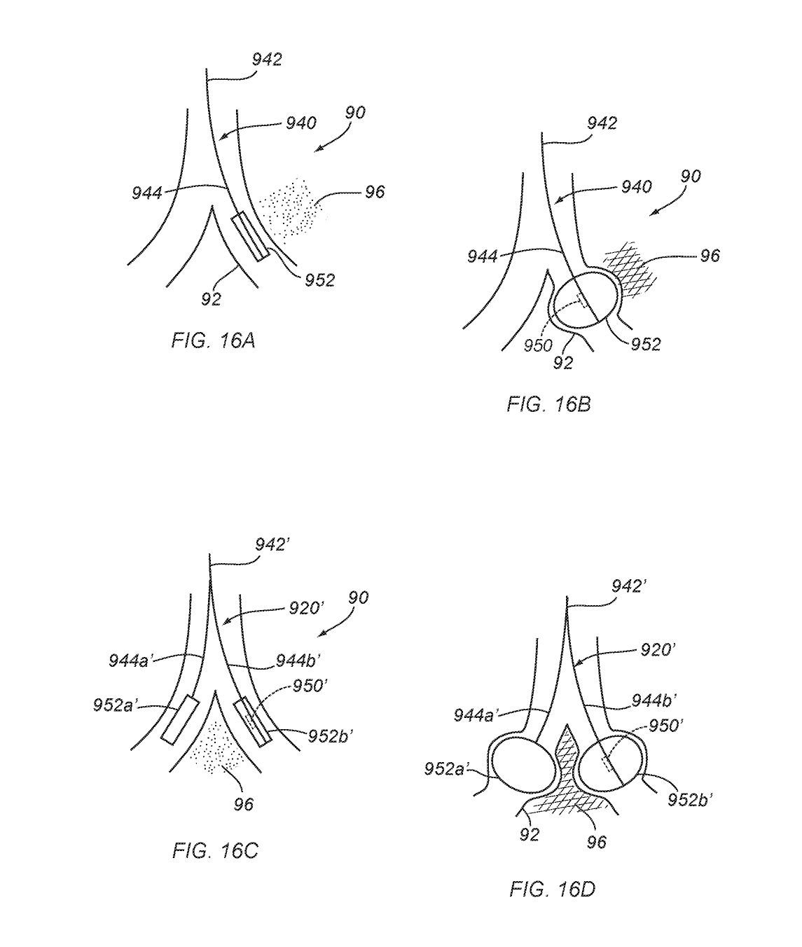

Turning to FIGS. 16A and 16B, another exemplary embodiment of an ultrasound imaging device 940 is shown that may be introduced into an airway 92 within a lung 90, e.g., to image tissue adjacent the airway 92, similar to other embodiments herein. Generally, the imaging device 940 includes a shaft or other elongate member 942 including a proximal end (not shown), a distal end or portion 944 sized for introduction into a body lumen, and an imaging transducer 950 carried on the distal portion 944. In addition, the distal portion 944 carries a balloon 952 thereon, e.g., surrounding the transducer 950, which may be used to compress tissue adjacent the body lumen and/or otherwise enhance imaging. The balloon 952 may be formed from compliant material, e.g., such that the size of the balloon 952 expands in proportion to the amount of fluid introduced into the balloon 952 and/or conforms to surrounding anatomy. Alternatively, the balloon 952 may be formed from semi-compliant or non-compliant material, e.g., such that the balloon 952 expands to a predetermined size and/or shape. A source of inflation media, e.g., an acoustic-coupling fluid (not shown), may be coupled to the proximal end of the imaging device 940 and communicate via a lumen (also not shown) with an interior of the balloon 952.

During use, the distal portion 944 may be introduced into an airway 92 with the balloon 952 collapsed, e.g., similar to other embodiments herein, until the transducer 950 is disposed adjacent to aerated lung tissue 96. The balloon 952 may then be inflated, e.g., with an acoustic-coupling fluid, until the balloon 952 distends the wall of the airway 92, thereby compressing the adjacent lung tissue 96 and reducing the air content within the tissue, which may improve the quality of ultrasound images of the lung tissue 96 obtained using the transducer 950.

FIGS. 16C and 16D show an alternative embodiment of an ultrasound imaging device 940′ that includes a shaft or other elongate member 942′ including a proximal end (not shown) and a distal portion that includes a first leg 944a′ and a second leg 944b,′ with each leg 944′ carrying a balloon 952.′ The second leg 944b′ may also include an imaging transducer 950,′ e.g., within an interior of the second balloon 952b.′ During use, the first and second legs 944′ may be introduced into the patient's body together with the balloons 952′ collapsed, e.g., using an access sheath, catheter, or other delivery device, similar to other embodiments herein. The legs 944′ may then be directed into adjacent branches of the airway 92, e.g., using separate stylets, guidewires, or other guide instruments (not shown), such that the balloons 952′ are disposed generally on opposite sides of a tissue region 96. The balloons 952′ may then be inflated, e.g., with an acoustic-coupling fluid, until the balloons 952′ distend the wall of the airway 92 and/or compress the lung tissue 96 between the branches, e.g., to reduce air content within the tissue, which again may improve the quality of ultrasound images of the lung tissue 96 obtained using the transducer 950′ carried on the second leg 944b.′

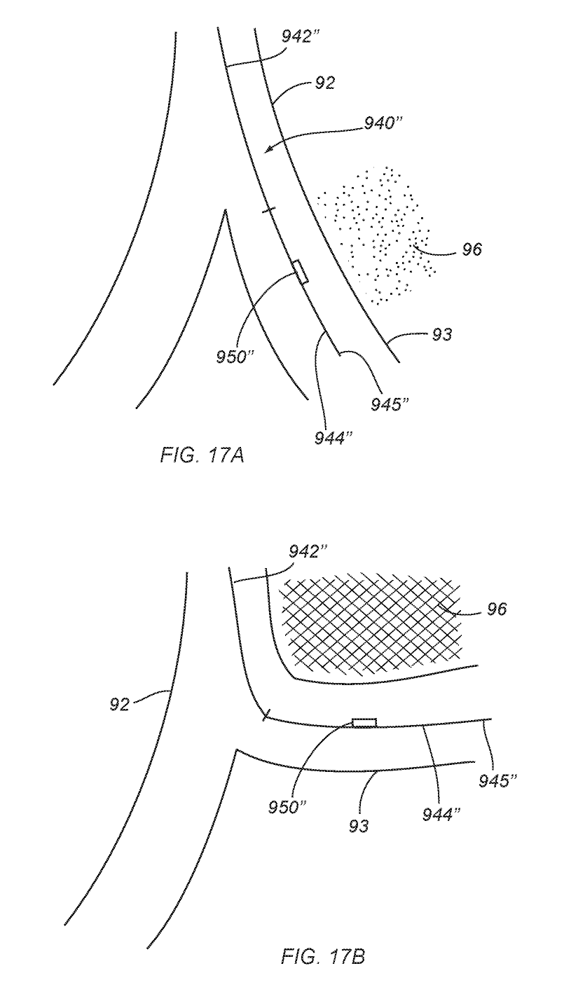

Turning to FIGS. 17A and 17B, yet another exemplary embodiment of an imaging device 940″ is shown that may be introduced into an airway 92 adjacent to aerated lung tissue 96. Generally, the imaging device 940″ includes a shaft 942″ including a proximal end (not shown) and a distal portion 944″ carrying an imaging element 950,″ e.g., similar to other embodiments herein. Unlike other embodiments, the distal portion 944″ may be steerable or otherwise directable between one or more shapes. For example, in a relaxed, straightened, or other first state, the distal portion 944″ may be introduced into the airway 92, e.g., using similar methods to other embodiments herein, and the imaging element 950″ may be positioned adjacent an area of interest adjacent the airway 92, e.g., including aerated lung tissue 96.

As shown in FIG. 17B, the distal portion 944″ may then be directed to a curved, bent, deflected, or other second state, thereby distorting a portion 93 of the airway 92 and compressing the adjacent lung tissue 96 to reduce the air content and improve ultrasound image quality. In an exemplary embodiment, the imaging device 940″ may include one or more steering wires, cables, or other elements (not shown) that are slidably received within a lumen in the distal portion 944″ and coupled adjacent the distal tip 945″ such that actuation of the steering element(s) causes the distal portion 944″ to bend to a desired shape. In another embodiment, a stylet or other pre-shaped member, e.g., having a bent, curved, or other pre-shaped distal region, may be inserted into a lumen of the imaging device 940″ and advanced into the distal portion 944″ to cause the distal portion 944″ to at least partially adopt the shape of the stylet.

In a further alternative, the distal portion 944″ may be biased to a bent, curved, or other deflected shape, and a stylet or other guide member may be inserted into the imaging device 940″ to at least partially straighten the distal portion 944″ and/or otherwise facilitate introduction of the distal portion 944″ into the airway 92. Once positioned as desired, the stylet may be removed, whereupon the distal portion 944″ may move towards its deflected shape to bend the portion 93 of the airway 92 and/or otherwise compress the aerated tissue 96. In this alternative, the stylet may be reintroduced after imaging and/or other procedures to facilitate withdrawal of the distal portion 944.″

In any of these embodiments, after imaging the tissue 96, a procedure may be performed within the airway 92. For example, a biopsy device (not shown) may be introduced into the airway 92 (e.g., via the same device used to introduce the imaging device 940″ or independently of the imaging device 940″) and used to perform a biopsy within the tissue 96 while imaging the tissue 96 and/or biopsy device.

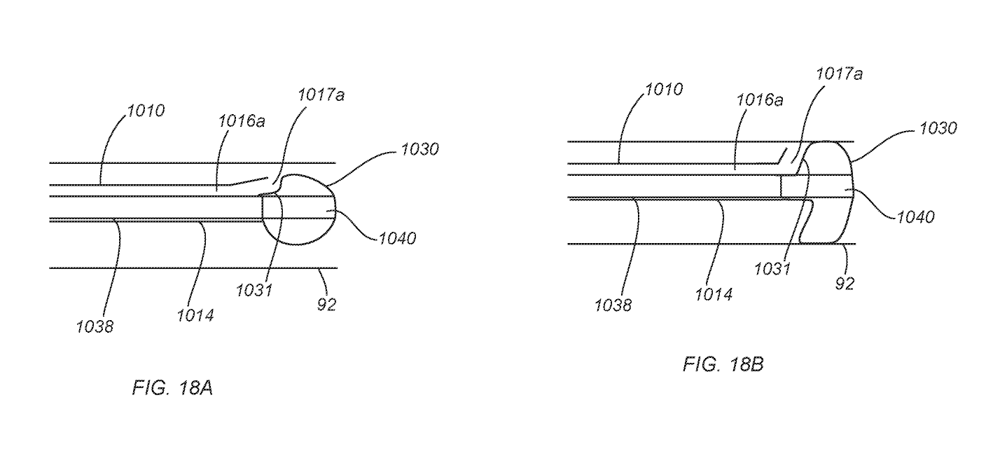

Turning to FIGS. 18A and 18B, still another exemplary embodiment of an access device or catheter 1010 is shown for performing a medical procedure, e.g., accessing, imaging, and/or performing a biopsy, similar to other embodiments herein. Generally, the catheter 1010 includes a proximal end (not shown), a distal end or portion 1014 sized for introduction into an airway or other body lumen 92, and one or more lumens 1016 extending between the proximal end and the distal portion 1014, generally similar to other embodiments herein. For example, the catheter 1010 includes a working channel or lumen 1016a communicating with an outlet 1017a in a side wall of the distal portion 1014, e.g., through which one or more biopsy devices may be advanced.

In addition, the catheter 1010 includes an imaging element, e.g., ultrasound transducer 1040, and a balloon 1030 surrounding or otherwise overlying the transducer 1040, both carried on the distal portion 1014. The balloon 1030 may be formed from materials such that the balloon 1030 is biased to a predetermined shape upon expansion, e.g., by molding the balloon material into the predetermined shape and/or including support materials within the material. The balloon 1030 may be coupled to an actuating element 1038, e.g., an elongate rod and the like, which may be manipulated to alter the shape of the balloon 1030 upon expansion. In addition, the balloon 1030 may be empty, partially filled, or completely filled with ultrasound-conductive fluid to modify the shape of the balloon 1030 and/or acoustically couple the transducer 1040 to surrounding tissue.

During use, the distal portion 1014 may be introduced into an airway 92, similar to other embodiments herein, and positioned adjacent an area of interest, e.g., while using the transducer 1040 to obtain images of the area. As desired, after partially or fully inflating the balloon 1030, the actuator element 1039 may be manipulated by the user to cause the balloon 1030 to change to a desired shape.

For example, a wall 1031 of the balloon 1030 may be disposed adjacent the outlet 1017a of the working lumen 1016a and may be supported to provide a ramped surface for guiding a biopsy device (not shown) advanced through the working lumen 10106a. For example, the actuating element 1038 may be manipulated to change the shape of the balloon 1030, thereby changing the orientation of the wall 1031 to a position desired by the user. In addition or alternatively, the wall 1031 of the balloon 1030 may include one or more materials whose properties may be modified, e.g., by applying energy thereto, to change a shape and/or rigidity of the wall 1031. For example, the wall 1031 may include piezoelectric material that may be actuated from the proximal end of the catheter 1010 to change the shape and/or rigidity of the wall 1031. This manipulation may facilitate real-time adjustment of the trajectory of a biopsy device placed in the working channel 1016a and deployed from the outlet 1017a, e.g., to access an area of interest visualized using ultrasound.

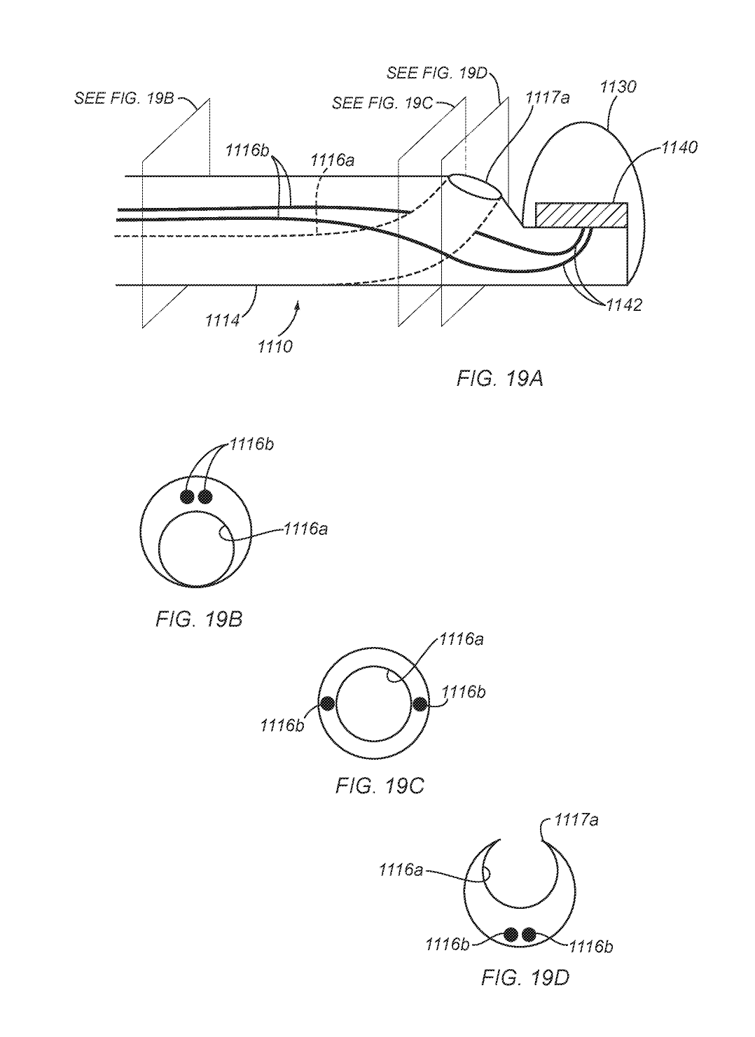

Turning to FIG. 19A, another exemplary embodiment of an access device or catheter 1110 is shown for performing a medical procedure, e.g., accessing, imaging, and/or performing a biopsy, similar to other embodiments herein. Generally, the catheter 1110 includes a proximal end (not shown), a distal end or portion 1114 sized for introduction into an airway or other body lumen, and one or more lumens 1116 extending between the proximal end and the distal portion 1114, generally similar to other embodiments herein. For example, the catheter 1110 includes a working channel or lumen 1116a communicating with an outlet 1117a in a side wall of the distal portion 1114, e.g., through which one or more biopsy devices may be advanced.

In addition, the catheter 1110 includes an imaging element, e.g., ultrasound transducer 1140, and a balloon 1130 surrounding or otherwise overlying the transducer 1040, both carried on the distal portion 1014, similar to other embodiments herein. In addition, the distal portion 1114 may also include a pair of lumens 1116b that receive respective wires or other conductive elements 1142 for operating the transducer 1140. As can be seen in FIGS. 19B-19D, the location of the wire lumens 1116b may change along the length of the distal portion 1114, e.g., to minimize a profile of the distal portion 1114 yet accommodate the wire lumens 1116b and the working lumen 1116a. For example, as can be seen in FIG. 19C, the wire lumens 1116b may be disposed on either side of the working lumen 1116a along a region of the distal portion 1114 and then may be disposed immediately adjacent one another, e.g., to accommodate the outlet 1117a of the working lumen 1116a. The wire lumens 1116b may extend separately to the proximal end of the catheter 1110 or may be merge into a single wire lumen (not shown) at a desired location along the length of the catheter 1110.

It should be noted that the present examples provide for use of the proposed embodiments and associated instruments in the airway to access adjacent parenchyma. However, such a catheter may also be used to access an area of interest in any area of the body that is adjacent to a lumen. This includes, but is not limited to the gastrointestinal system, biliary system, pancreas, and the cardiovascular system. It should further be reiterated that any of the exemplary embodiments may be utilized alone or in combination with each other to create further novel embodiments.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A system for performing a procedure within a patient's lung, comprising:
an elongate tubular member comprising a proximal portion, a distal portion sized for introduction into a body lumen of a lung, and one or more lumens extending between the proximal and distal portions, thereby defining a longitudinal axis;
an ultrasound imaging device deployable from the distal portion for imaging tissue adjacent the body lumen;
an expandable member on the distal portion configured to expand within the body lumen to isolate a region of the body lumen beyond the distal portion; and
a source of vacuum coupled to the proximal portion and communicating via a lumen of the tubular member with a port in the distal portion for aspirating fluid within region of the body lumen beyond the distal portion to collapse the body lumen around the imaging device.

2. The system of claim 1, further comprising a source of ultrasound-conductive fluid coupled to the proximal portion and communicating via a lumen of the tubular member with a port in the distal portion for delivering the ultrasound-conductive fluid into the body lumen to enhance acoustic coupling of the imaging device with tissue surrounding the body lumen.

3. The system of claim 1, further comprising a biopsy device deployable from the distal portion for performing a biopsy within tissue adjacent the body lumen.

4. The system of claim 3, wherein the biopsy device comprises a needle disposed within an instrument lumen of the tubular member, the instrument lumen comprising a ramped surface within the distal portion for directing a tip of the needle laterally relative to the longitudinal axis.

5. The system of claim 1, wherein the tubular member comprises an instrument lumen comprising an outlet on a distal tip of the distal portion, and wherein the imaging device is deployable from the outlet substantially parallel to the longitudinal axis.

6. The system of claim 5, wherein the imaging device further comprises an expandable member configured to be expanded when the imaging device is deployed from the outlet to substantially center the imaging device within the body lumen.

7. A system for performing a biopsy within a patient's lung, comprising:
an elongate tubular member comprising a proximal portion, a distal portion sized for introduction into a body lumen of a lung, and a plurality of lumens including a biopsy lumen and an imaging lumen extending between the proximal and distal portions, thereby defining a longitudinal axis;
an ultrasound imaging device disposed within the imaging lumen and deployable from an outlet in the distal portion substantially parallel to the longitudinal axis for imaging tissue adjacent the body lumen;
a needle disposed within the biopsy lumen of the tubular member, the biopsy lumen comprising a ramped surface within the distal portion adjacent a side port for directing a tip of the needle laterally relative to the longitudinal axis out the side port;
an expandable member on the distal portion proximal to the side port and configured to expand within the body lumen to isolate a region of the body lumen beyond the distal portion; and
a source of vacuum coupled to the proximal portion and communicating via a lumen of the tubular member with a vacuum port in the distal portion for aspirating fluid within region of the body lumen beyond the distal portion to collapse the body lumen around the imaging device.

8. The system of claim 7, wherein the source of vacuum communicates via one of the biopsy lumen, the imaging lumen, and a vacuum lumen separate from the biopsy lumen and the imaging lumen.

11. A method for performing a procedure within a patient's lung, comprising:
introducing a distal portion of an elongate tubular member into a body lumen of a lung;
deploying an ultrasound imaging device from the distal portion within the body lumen;
expanding an expandable member on the distal portion within the body lumen to isolate a region of the body lumen beyond the distal portion;
aspirating fluid within the body lumen via the tubular member to at least partially collapse the body lumen around the imaging device; and
activating the imaging device to identify a target tissue site adjacent the body lumen.

12. The method of claim 11, further comprising delivering an ultrasound conductive fluid via the tubular member into the body lumen to enhance acoustic coupling of the imaging device with tissue surrounding the body lumen.

13. The method of claim 11, further comprising deploying a biopsy device from the distal portion to perform a biopsy at the target tissue site.

14. The method of claim 13, wherein the biopsy device comprises a needle and wherein deploying a biopsy device comprises advancing the needle from the distal portion laterally relative to the longitudinal axis into the target tissue site.

15-20. (canceled)

21. A system for accessing a body lumen within a patient's lung, comprising:
a bronchoscope comprising a shaft sized for introduced into a patient's lung and a working channel; and
an access sheath comprising a proximal portion, a distal portion, and one or more lumens extending therebetween, at least the distal portion being expandable from a contracted condition sized for introduction into the working channel of the bronchoscope to an expanded condition larger than the shaft.

22. The system of claim 21, wherein the distal portion of the access sheath is biased to the expanded condition and resiliently compressible to the contracted configuration.

23. The system of claim 21, wherein the distal portion of the access sheath comprises one or more creases extending along a length of the distal portion for folding or rolling the distal portion into the contracted configuration.

24. The system of claim 21, wherein the one or more lumens comprise a working lumen having an inner diameter in the expanded configuration that is larger than an inner diameter of the working channel of the bronchoscope.

25. The system of claim 21, wherein the one or more lumens comprise a working lumen having an inner diameter in the expanded configuration that is larger than an outer cross-section of the shaft of the bronchoscope.

26. The system of claim 21, wherein the distal portion comprises a plurality of longitudinal struts extending along the distal portion, a plurality of transverse struts coupled to adjacent longitudinal struts, and a membrane covering the longitudinal and transverse struts to define a working lumen in the expanded configuration, at least the transverse struts configured to be reoriented to allow the membrane to be folded or rolled to direct the distal portion to the contracted configuration.

27-37. (canceled)
